(19) United States
(12) Patent Application Publication
(10) Pub. No.: US 2018/0126158 A1
(43) Pub. Date: May 10, 2018

(54) Systems and Methods for Functional Restoration and Rehabilitation of Posture, Gait and Movement

(71) Applicant: Lyriq, LLC, St. Louis, MO (US)

(72) Inventors: Raul E. Perez, St. Louis, MO (US); Bevil Hogg, Murrieta, CA (US)

(21) Appl. No.: 15/682,993

(22) Filed: Aug. 22, 2017

Related U.S. Application Data
(60) Provisional application No. 62/377,744, filed on Aug. 22, 2016.

(51) Int. Cl. A61N 1/36

(52) U.S. Cl. CPC A61N 1/36003; A61N 1/3603

(57) ABSTRACT

A wearable electrical stimulation system for improving a motor function of a patient after the patient has a stroke. The system is deployed by positioning one stimulation unit on the head patient in electrical communication with a first portion of the patient's brain, positioning a second stimulation unit on a region of the patient comprising muscles requiring motor function improvement, and using a controller to activate the first stimulation unit and second stimulation unit. Upon activation, the first stimulation unit applies electrical stimulation to the first portion of the patient's brain and upon activation the second stimulation unit applies electrical stimulation to the first region of the patient. A system comprising a plurality of sensors for monitoring and improving the functions such as posture, movement, gait of a limb or a body portion is also disclosed.

### CROSS-REFERENCE

The present application relies on, for priority, U.S. Patent Provisional Application No. 62/377,744, entitled “Systems and Methods for Post-Stroke Rehabilitation and Improved Posture Management” and filed on Aug. 22, 2016.

The above-mentioned application is herein incorporated by reference in its entirety.

### FIELD

The present specification relates generally to systems and methods of delivering electrical stimulation to a user that may need physical functional restoration or that may need feedback and training corresponding to correct posture, gait, movement or coordination. In particular, electrical stimulation is delivered to a predetermined area of a user's anatomy comprising at least one limb or body portion of the user. More particularly, the present specification relates to wearable electrical stimulation devices that are programmable and monitorable using a mobile handheld device, and are programmed to stimulate a patient's nerves in a manner that addresses the issues of functional restoration and post-stroke rehabilitation and also generates coaching feedback to improve gait and correct posture.

### BACKGROUND

After a cerebrovascular trauma or accident, more commonly known as a stroke, people often suffer from resulting motor deficits including decreases in muscle activation, increases in muscle stiffness, and changes in gait patterns. In the past, the majority of research efforts were directed toward treatment of acute stroke: re-establishing perfusion of the affected areas and minimizing neuronal damage. With the advance of acute stroke treatment, the issues of functional restoration and post-stroke rehabilitation have become increasingly important.

Muscle movement is caused by electrical impulses originating in the brain, which are transmitted via nerve cells to the muscles. When a person wishes to initiate muscle movement, the brain sends electrical signals to the muscles. Upon arrival of these signals, the muscles respond by contracting. These electrical signals can be measured over muscles and they are called electromyographic (EMG) signals. When brain damage is caused by a stroke, regular electrical impulses are not generated or can no longer reach muscles, normal muscle contraction becomes impossible. Although there are always minuscule EMG signals, these are mostly extremely small and unable to control the muscles. This often leads to irreversible damage and loss of muscle function, resulting in muscle paralysis such as “claw” hand or “drop foot”.

After a stroke, the stroke survivor is rehabilitated by physical therapy. This is done in the hope that there will be some form of spontaneous recovery and that the muscles will not become too stiff. In most cases however, recovery does not occur and stroke survivors are left with severe disuse muscle atrophy and paralyzed muscles, greatly affecting their quality of life.

Patients suffering from Parkinson's disease often exhibit Parkinsonian gait. This disorder is exhibited by motor deficits, such as, but not limited to, shuffling steps and a general slowness of or total loss of movement. There are different forms of treatment available for Parkinsonian gait, including pharmaceutical agents such as L-dopa, external sensory cues such as auditory and visual cues, Deep Brain Stimulation (DBS) in various regions of the brain stem, and regular physiotherapy and exercises. However, known treatments often address the disorder either temporarily and/or partially.

The paralysis of muscles can often be prevented if the stroke survivor or a Parkinson's disease patient is offered a possibility to re-learn the use of the affected muscles by improving electrical muscle activity. There is, therefore, a need for systems and methods that stimulate a patient's brain and/or the affected limbs or body portions or regions in order to improve gait, functional ability, and motor function in the patient who may have sustained a stroke or has been affected by Parkinsonian gait. There is also a need for wearable neuro-stimulation devices that provide therapeutic feedback and/or stimulation to a user regarding a behavior of his limb or body portion that may or may not be affected, dysfunctional or diseased. The feedback along with positional, behavioral or movement map of the limb or body portion is utilized for persons such as, but not limited to, joggers, horseback riders, gymnasts, and dancers for whom correct posture, gait or bodily movement or coordination is vital.

### SUMMARY

The following embodiments and aspects thereof are described and illustrated in conjunction with systems, tools and methods, which are meant to be exemplary and illustrative, and not limiting in scope. The present application discloses numerous embodiments.

The present specification is directed towards a method for monitoring and improving the functions such as the posture and/or the movement and/or the gait of a limb or body portion of a user comprising: positioning a plurality of body sensors on a limb or body portion to assess at least one of the said of said limb or body portion; receiving the information recorded by said body sensors; comparing said information with reference parameters corresponding to similar functions; and providing real time feedback to the user based on said comparison.

The present specification discloses a method for improving a motor function of a patient after said patient has a stroke, comprising: positioning at least one first stimulation unit on the patient, wherein said at least one first stimulation unit is placed in electrical communication with a first region of the patient, said first region comprising a plurality of muscles requiring improvement of said motor function; acquiring, via a controller located in a mobile device external to said at least one first stimulation unit, first signals generated by said at least one first stimulation unit, said first signals representative of a plurality of body parameters associated with said first region of the patient; comparing said first signals with reference signals; and, performing at least one of the following actions if said first signals deviate from said reference signals beyond a threshold: activating said at least one first stimulation unit to apply electrical stimulation to said first region and, triggering feedback for the patient.

Optionally, said plurality of body parameters comprise data representative of at least one of position, posture, gait and movement of said first region of the patient.

Optionally, said reference signals are acquired by said controller from at least one second stimulation unit placed in electrical communication with a plurality of muscles not requiring improvement of said motor function in a second region of the patient. The first region may be a limb on a right side of the patient's body, and the second region may be a corresponding limb on a left side of the patient's body. The first region may be a limb on a left side of the patient's body, and the second region may be a corresponding limb on a right side of the patient's body.

Optionally, said reference signals are generated from at least one second stimulation unit placed in electrical communication with a plurality of muscles in a second region of a healthy person, wherein said healthy person is defined as an individual with muscles that do not have an impaired motor function. The first region may be a limb on a right side of the patient's body, and the second region may be a corresponding limb on a right side of the healthy person's body. The first region may be a limb on a left side of the patient's body, and the second region may be a corresponding limb on a left side of the healthy person's body.

Optionally, the method further comprises positioning a head stimulation unit on a head of the patient, wherein the head stimulation unit is placed in electrical communication with a first portion of a brain of the patient. The at least one first stimulation unit and the head stimulation unit may be activated synchronously. The at least one first stimulation unit and the head stimulation unit may be activated asynchronously.

Optionally, the plurality of muscles are in a limb in a right side of the patient's body and said first portion is in a left side or right side of the brain. Optionally, the plurality of muscles are in a limb in a left side of the patient's body and said first portion is in a right side or left side of the brain.

The present specification also discloses a method for improving a function of a patient, comprising: positioning at least one first stimulation unit on the patient, wherein said at least one first stimulation unit is placed in electrical communication with a first region of the patient, said first region comprising at least one limb of said patient; acquiring, via a controller located in a mobile device external to said at least one first stimulation unit, first signals generated by said at least one first stimulation unit, said first signals representative of a reference model or map of said first region of the patient, wherein said reference model or map defines a baseline position or movement of said first region and said first signals are acquired while said first region is in a first state; acquiring, via the controller, second signals generated by said at least one first stimulation unit, wherein said second signals are acquired while said first region performs said function; comparing said first signals with second signals to determine a deviation of position or movement of said first region from said baseline position or movement; and performing at least one of the following actions if said first signals deviate from said second signals beyond a threshold: activating said at least one first stimulation unit to apply electrical stimulation to said first region and triggering a feedback for the patient.

Optionally, first state is representative of said first region performing said function, and said function is at least one of a reference position, posture, gait and movement of said first region.

Optionally, the method further comprises positioning a second stimulation unit on a head of the patient, wherein the second stimulation unit is placed in electrical communication with a first portion of a brain of the patient. The first stimulation unit and second stimulation unit may be activated synchronously or asynchronously.

Optionally, the at least one limb comprises a plurality of muscles requiring a restoration of function after said patient has one of a stroke and a Parkinsonian gait. Optionally, the at least one limb comprises a healthy limb. The first region may be in a right side or a left side of the patient's body and said first portion may be in a right side or a left side of the brain.

The present specification also discloses a method for improving an impaired motor function of a patient with Parkinsonian gait, comprising: positioning at least one first stimulation unit over skin covering one or more target regions of the patient's body, wherein the one or more target regions are at least partially responsible for the impaired motor function of the patient; pairing said at least one first stimulation unit with a remote management device; and operating the remote management device to: acquire target signals generated by said at least one first stimulation unit to produce a first map indicative of the impaired motor function; acquire reference signals generated by at least one second stimulation unit to produce a reference map of said one or more target regions; compare said first map and reference map; and based on said comparison, perform at least one of triggering said at least one first stimulation unit to apply electrical stimulation to said one or more target regions and triggering a feedback for the patient.

Optionally, the positioning of at least one first stimulation unit comprises positioning at least two stimulation units, which are activated synchronously or asynchronously.

Optionally, the one or more target regions comprises at least one of a big toe, a knee, an ankle, and a joint of the patient.

Optionally, said one or more target regions are on one side of the patient's body, and said at least one second stimulation unit is positioned on one or more regions on another side of the patient's body, said one or more regions on another side of the patient's body not being at least partially responsible for the impaired motor function of the patient.

Optionally, said at least one second stimulation unit is positioned on one or more healthy regions of a healthy individual, said one or more healthy regions not being at least partially responsible for the impaired motor function of the patient.

Optionally, at least one of triggering said at least one first stimulation unit to apply electrical stimulation to said one or more target regions and triggering a feedback for the patient is triggered only if said first map deviates from said reference map more than a predefined threshold.

The present specification also discloses a system for monitoring and improving at least one motor function of a user, wherein said at least one motor function comprises at least one of posture, movement and gait of a limb or body portion, comprising: a plurality of body sensors positioned on the limb or body portion to assess said at least one motor function, wherein said limb or body portion is of a first side of the user's body; and a control unit adapted to receive information recorded by said body sensors, compare said information with reference parameters corresponding to said at least one motor function, and provide real time feedback to the user based on said comparison.

Optionally, the system further comprises a display and said control unit is adapted to receive, and transmit to the display, a plurality of media and wherein said media provides real time coaching in a form of suggestions or recommendations to improve said at least one motor function.

Optionally, the system further comprises a remote server on which the information recorded by said control unit is stored.

Optionally, the said body sensors comprise at least one of an accelerometer, inclinometer and gyroscope.

Optionally, the control unit is at least one of a hand-held device, mobile phone, and tablet computer.

Optionally, the system further comprises a display and said feedback is provided in the form of a visual, audio, and/or vibratory alarm on said display.

Optionally, the user is provided with a progress report comprising his progress with respect to other individuals.

Optionally, the system further comprises a plurality of body sensors positioned on a corresponding limb or body portion on a second side of the user's body. The reference parameters may be derived in real time from said plurality of sensors positioned on the corresponding limb or body portion on the second side of the user's body.

Optionally, the system further comprises a body sensor positioned on a head of the user to monitor at least one of a posture, position, gait and movement of the head.

Optionally, said reference parameters are pre-defined. Optionally, said reference parameters are estimated by stimulating said functions of the user in a control environment. Optionally, said reference parameters are derived from data recorded for said functions for another individual. Optionally, said reference parameters are derived from data recorded for said functions for a set of people over a period of time.

Optionally, said system is used by stroke patients to improve a function of an affected limb or body part.

Optionally, said system is used by sportsmen to improve the posture/movement/gait corresponding to their limb or body portion.

The present specification also discloses a method for monitoring and improving functions such as the posture and/or the movement and/or the gait of a limb or body portion of a user comprising: positioning a plurality of body sensors on a limb or a body portion to assess at least one of said functions of said limb or body portion; receiving the information recorded by said body sensors; comparing said information with reference parameters corresponding to similar functions; and providing real time feedback to the user based on said comparison.

The aforementioned and other embodiments of the present specification shall be described in greater depth in the drawings and detailed description provided below.

### BRIEF DESCRIPTION OF THE DRAWINGS

These and other features and advantages of the present specification will be further appreciated, as they become better understood by reference to the following detailed description when considered in connection with the accompanying drawings:

FIG. 1A is a block diagram of a system for stroke rehabilitation and/or posture restoration and management, in accordance with an embodiment of the present specification;

FIG. 1B is a block diagram of a system for delivering stimulation and generating positional feedback in relation to a targeted limb or body portion, in accordance with an embodiment of the present specification;

FIG. 2A illustrates placement and functioning of at least one stimulation unit, in accordance with some embodiments of the present specification;

FIG. 2B illustrates placement and functioning of an optional head module and at least one stimulation unit, in accordance with some embodiments of the present specification;

FIG. 3A is a flowchart illustrating a plurality of exemplary steps of a method of delivering stimulation to treat at least one targeted limb or body portion, in accordance with some embodiments;

FIG. 3B is a flowchart illustrating a plurality of exemplary steps of another method of delivering stimulation to treat at least one targeted limb or body portion, in accordance with some embodiments;

FIG. 3C illustrates placement and functioning of at least one reference stimulation unit and at least one stimulating stimulation unit on a patient, in accordance with some embodiments of the present specification;

FIG. 3D illustrates placement and functioning of at least one reference stimulation unit on an unaffected individual and at least one stimulating stimulation unit on a patient, in accordance with some embodiments of the present specification;

FIG. 4A is a flowchart illustrating a plurality of exemplary steps of a method of monitoring and recording a movement, position, posture, bearing or gait of a user's targeted limb or body portion that may or may not be affected, dysfunctional, diseased or errant, in accordance with some embodiments;

FIG. 4B is illustrates normal leg movement in comparison with impaired leg movement for generating reference maps in accordance with some embodiments of the present specification;

FIG. 5A is a flowchart illustrating a plurality of exemplary steps of a method of monitoring and recording a movement, position, posture, bearing, behavior or gait of the user's targeted limb or body portion that may be affected, dysfunctional, diseased or errant, in accordance with some embodiments;

FIG. 5B is a flowchart illustrating exemplary steps of a method of delivering stimulation to correct Parkinsonian gait, in accordance with some other embodiments;

FIG. 5C illustrates normal footsteps in comparison with impaired, shortened footsteps for generating reference maps in accordance with some embodiments of the present specification;

FIG. 6 illustrates a system for performance feedback and coaching in accordance with an embodiment of the present specification;

FIG. 7 illustrates bilateral sensors positioned to capture and compare relative movement of the corresponding limbs or body portions in accordance with an embodiment;

FIG. 8 illustrates a flow chart depicting the steps followed in using the bilateral sensors positioned on corresponding limbs or body portions in accordance with an embodiment of the present specification;

FIG. 9 illustrates a flow chart depicting the steps followed in evaluating the performance of a specific limb or body part against a reference model/map or expected performance level;

FIG. 10 illustrates a flow chart depicting the steps followed to estimate the reference parameters in a control environment.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

In the description and claims of the application, each of the words “comprise” “include” and “have”, and forms thereof, are not necessarily limited to members in a list with which the words may be associated.

As used herein, the indefinite articles “a” and “an” mean “at least one” or “one or more” unless the context clearly dictates otherwise.

While the specification has been generally described in the context of treating defects in the posture and mobility of patients who may have suffered from stroke, the various embodiments described herein are applicable to other disorders that affect posture and mobility, such as but not limited to patients with Parkinsonian gait. All references made to patients who may have suffered from stroke are therefore also applicable to patients with Parkinsonian gait.

### Post-Stroke Rehabilitation and Posture Restoration System

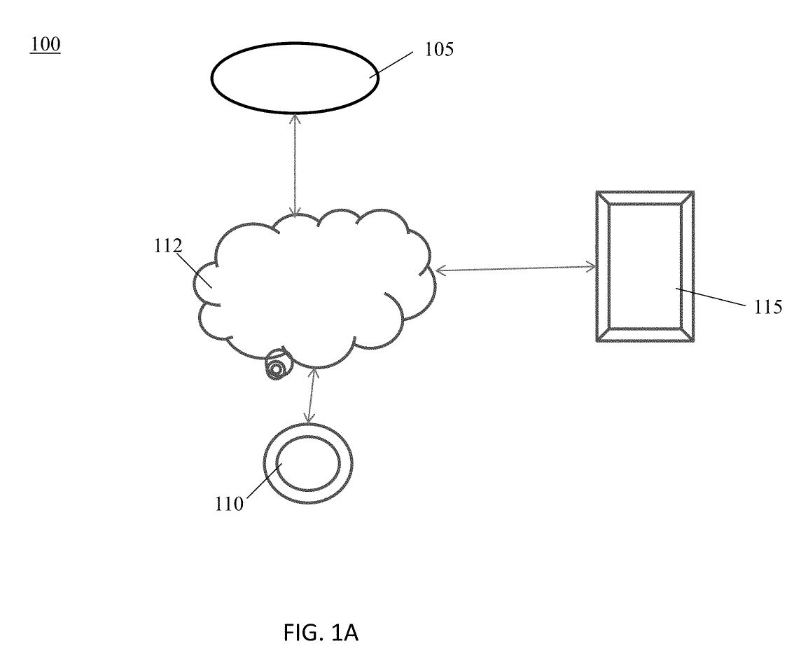

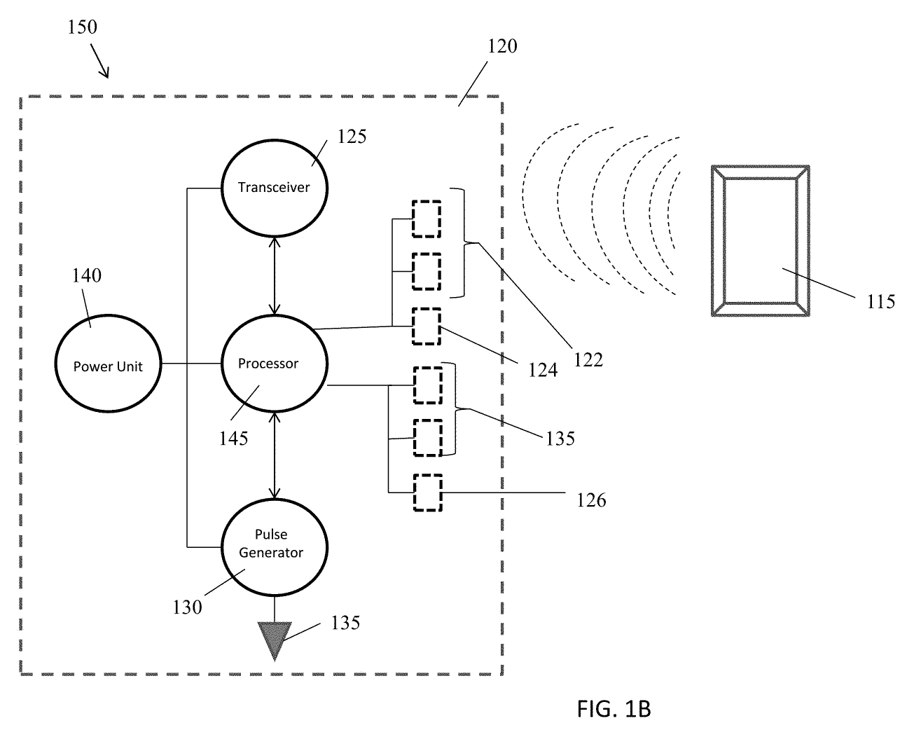

FIG. 1A illustrates a system 100 for stroke rehabilitation and/or posture restoration and management, in accordance with an embodiment of the present specification. The system 100 comprises an optional head module 105, at least one stimulation unit 110 and a remote management device 115. The optional head module 105 is configured to be worn, positioned, mounted or placed on a user's head while the at least one stimulation unit 110 is configured to be worn, positioned, mounted or placed on the user's targeted limb or body portion. It should be appreciated that use of the head module 105 is optional, in accordance with aspects of the present specification, such that a treatment of targeted limbs or body portions is achieved by the placement of one or more stimulation units 110 on the user's one or more affected limbs and/or treatment regions. It should be appreciated that, in various embodiments, the optional head module 105 and the at least one stimulation unit 110 are worn or positioned using means such as adhesive, belt or strap. In some embodiments, the targeted limb or body portion may be weak, paralyzed, errant, diseased, compromised or dysfunctional due to neurological stroke or trauma, or due to Parkinson's disease (PD), for example. In some embodiments, the targeted limb or body portion may be healthy or normal yet requiring behavioral, postural or movement related monitoring, feedback and training.

In various embodiments, the optional head module 105, the at least one stimulation unit 110 and the remote management device 115 are in data communication with each other using any one or a combination of cellular, Internet, TCP/IP, Ethernet, Bluetooth, wired, or wireless network 112. In one embodiment, the optional head module 105, the at least one stimulation unit 110 and the remote management device 115 are in wireless data communication with each other.

The remote management device 115 comprises a computer readable medium and processor and can be any type of computing and communication device known to persons of ordinary skill in the art such as, but not limited to, a computer, server, mobile phone, gateway, laptop, desktop computer, netbook, Ultrabook, personal data assistant, remote control device or any other device capable of accessing a cellular, Internet, TCP/IP, Ethernet, Bluetooth, wired, or wireless network. In preferred embodiments, the remote management device 115 is a hand-held computing device such as a smartphone, PDA, or tablet. In various embodiments, the remote management device 115 runs or implements a Rehabilitation and Restoration software application (hereinafter referred to as ‘RRA’) that activates, deactivates and controls the optional head module 105 as well as the at least one stimulation unit 110 to provide a plurality of stimulation therapies and/or posture or gait management coaching and feedback.

In various embodiments, the optional head module 105 and the at least one stimulation unit 110 are all wearable units that can be used independently (as a stand-alone single unit) or in conjunction with multiple other similar units.

In accordance with various embodiments, the optional head module 105 and the at least one stimulation unit 110, collectively referred to hereinafter as ‘therapy devices’ 150, as shown in FIG. 1B, have an enclosure or casing 120 comprising a transceiver 125 electronically connected to a microprocessor or microcontroller 145 wherein the transceiver 125 enables wireless communication with the remote management device 115, a pulse generator 130 to generate a plurality of electrical pulses for application through one or more sensor electrodes 135 and a power unit 140. The enclosure or casing 120 also comprises one or more actuators 122 such as push buttons or switches to switch the therapy devices 150 on/off and to enable user control or settings, at least one visual indicator 124, such as an LED (Light Emitting Diode), and one or more tactile and audio indicators 126, such as a vibrator, buzzer or beeper to provide feedback to the user. It should also be appreciated that, in one embodiment, the therapy devices 150 comprise no such actuators 122 and are entirely controlled by the remote management device 115.

In various embodiments, the microprocessor 145 is in electronic communication with one or more sensor electrodes 135 such as, but not limited to, an accelerometer, an inclinometer, an impedance and/or a bio-impedance sensor. In accordance with an aspect of the present specification, the one or more electrodes 135 comprise transcutaneous electrodes for placement on the user's epidermal surface and to deliver electrical stimulation therapy to the user. In some embodiments, the one or more sensor electrodes 135 may differ, in terms of the types of sensors, between the optional head module 105 and the at least one stimulation unit 110. For example, in some embodiments, the accelerometer and/or inclinometer is included in the at least one stimulation unit 110 and not included in the optional head module 105.

### Recovery and Rehabilitation

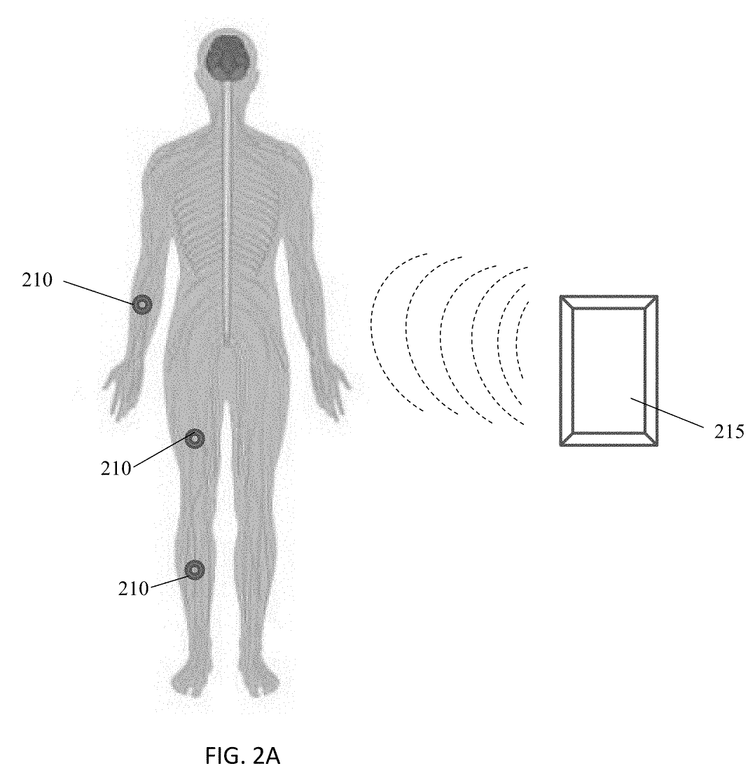

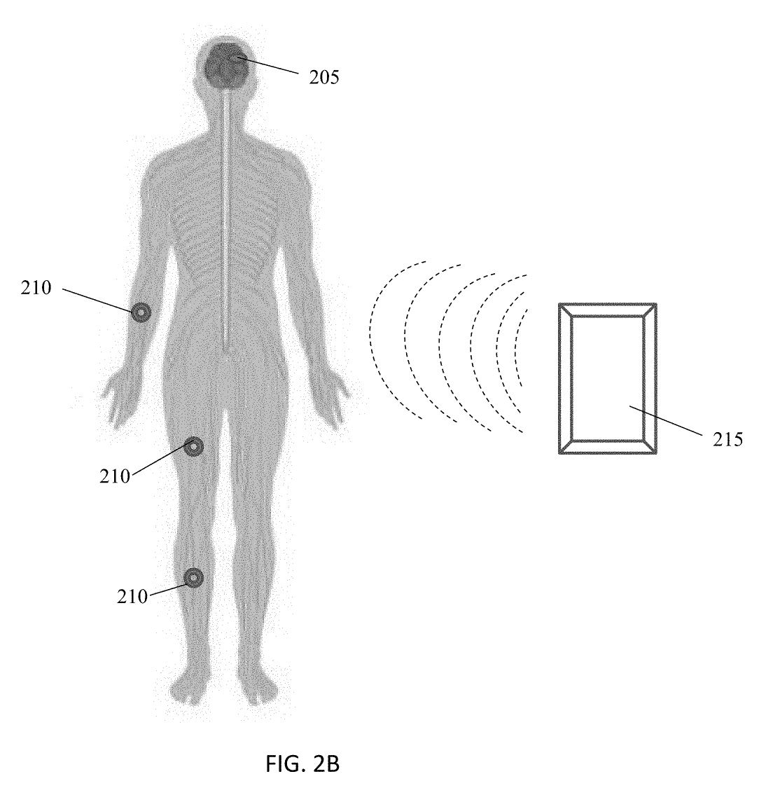

In accordance with some aspects of the present specification, the therapy devices comprising at least one stimulation unit and an optional head module, in data communication with the remote management device, deliver stimulation therapy to the user's one or more targeted limb or body portion that may or may not be dysfunctional, paralyzed, errant, affected, diseased or weakened and optionally to the user's brain. Referring now to FIG. 2A, in some embodiments, to treat one or more targeted limbs or body portions one or more stimulation units 210 are placed on the user's one or more affected limbs, such as on an arm and/or a leg as illustrated in the figure, to deliver stimulation therapy to the affected limbs. In other embodiments, as shown in FIG. 2B, to treat one or more targeted limbs or body portions a head module 205 is also placed on the user's head to deliver stimulation therapy to the brain in addition to one or more stimulation units 210 placed on the user's one or more affected limbs. In an embodiment, a patient suffering from Parkinsonian gait may have one or more stimulation units 210 placed at one or more associated target regions such as under one or both feet, under at least one big toe, near one or both ankles and/or knees, or any other joint, which may affect the mobility of the patient. It should be appreciated that use of the head module 205 is optional, in accordance with aspects of the present specification, such that a treatment of targeted limbs or body portions is achieved by the placement of one or more stimulation units 210 on the user's one or more affected limbs and/or treatment regions. As shown in FIGS. 2A and 2B, a remote management device 215, such as a smartphone, running the rehabilitation and restoration software application (RRA) controls and coordinates delivery of stimulation therapy across the optional head module 205 (FIG. 2B) and the stimulation units 210.

It should be appreciated that, in some embodiments, the RRA is programmed (by the user or by a medical personnel, physician, physiotherapist, trainer or coach) to deliver stimulation therapy automatically, according to a predefined schedule, or in response to triggering events (such as, but not limited to, a specific posture, gait or errant behavior of the targeted limb). Alternatively, in some embodiments, the optional head module and/or the stimulation units could be activated either manually by the user or triggered by specific posture/gait feedback obtained from accelerometer or inclinometer built into the optional head module and/or the stimulation units.

With reference to FIG. 2B, in some embodiments, placement sites of the head module 205 on the user's head for brain stimulation and of the stimulation units 210 on the affected limbs are contralateral. That is, if the targeted limbs or body regions correspond to the right side of the user's body then the stimulation units 210 are positioned on the targeted limbs or body regions on the right side while the head module 205 is positioned to stimulate the left side of the user's brain. Similarly, if the targeted limbs or body regions correspond to the left side of the user's body then the stimulation units 210 are positioned on the targeted limbs or body regions on the left side while the head module 205 is positioned to stimulate the right side of the user's brain.

In other embodiments, placement sites of the head module 205 on the user's head for brain stimulation and of the stimulation units 210 on the targeted limbs are ipsilateral. That is, the stimulation units 210 are positioned on either side (left and right sides) of the body while the head module 205 is positioned on the user's head so as to stimulate the cerebellum portion of the brain. Such balanced stimulation is particularly intended for a user who may have targeted limbs or body portions on either side of their body and/or for a user who may have body balancing issues due to targeted limbs or body portions on any one or both sides of their body.

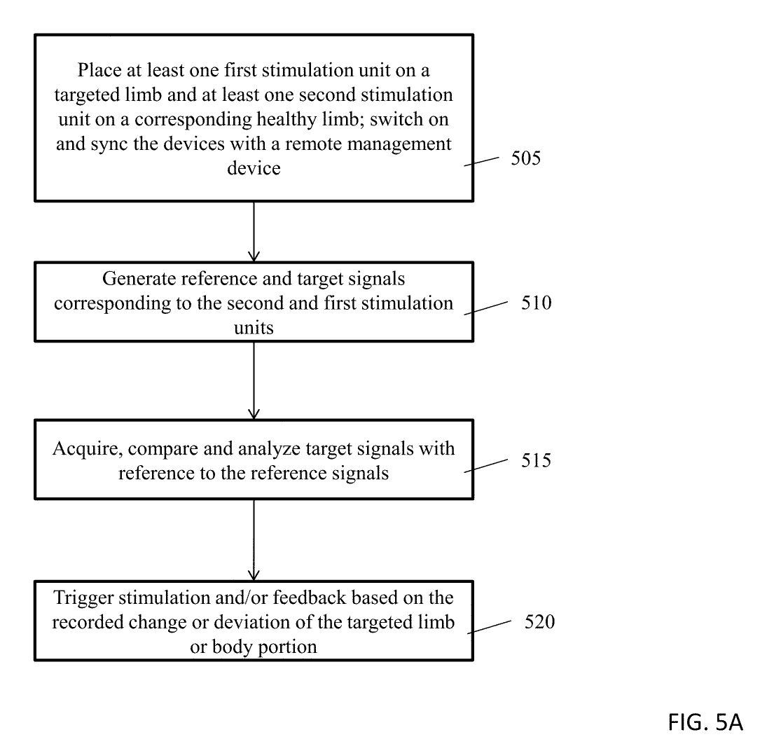

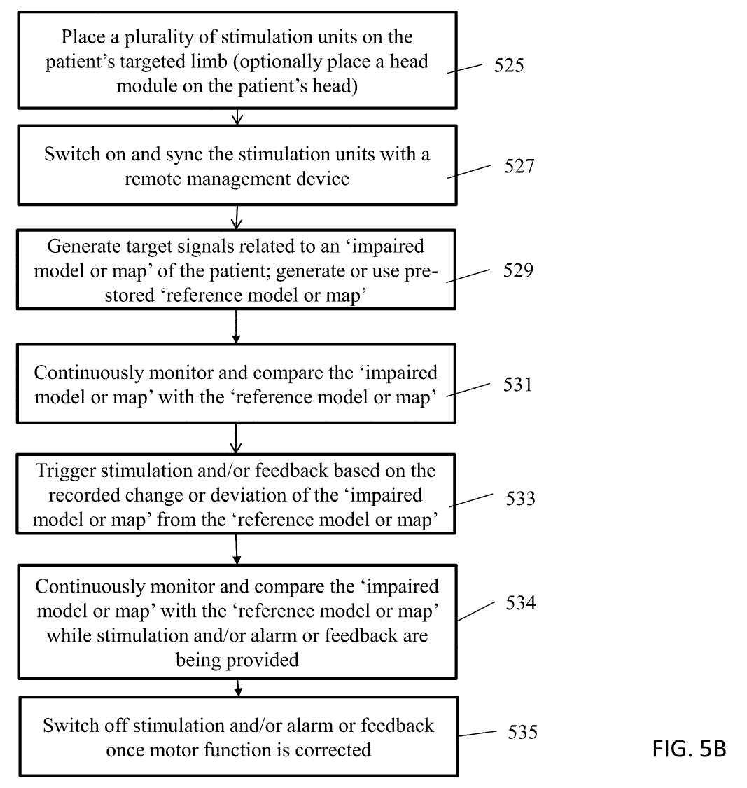

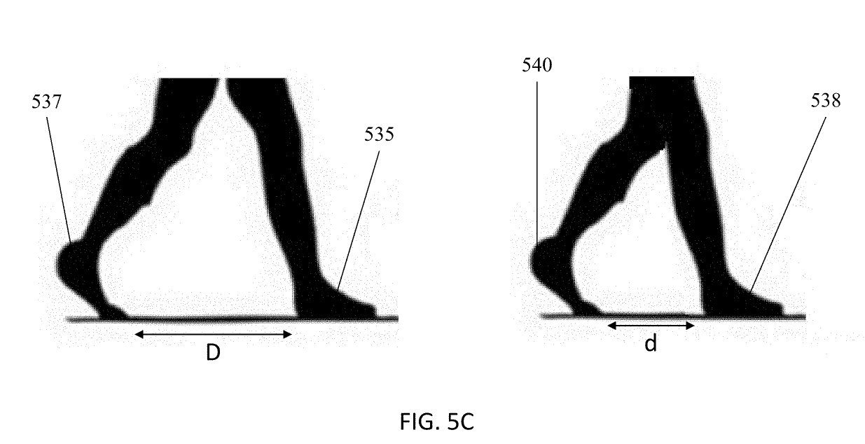

Referring to FIG. 2A, in some embodiments the stimulation units 210 are triggered by the RRA, running on the remote management device 215, to stimulate the respective sites synchronously. In other words, a stimulation session is activated and coordinated by the RRA to deliver stimulation therapy simultaneously to the targeted limbs. In alternate embodiments, the stimulation units 210 stimulate the respective sites asynchronously. That is, a stimulation session is activated and coordinated by the RRA such that the stimulation units 210 do not deliver stimulation to the respective sites simultaneously. Thus, in some embodiments, a stimulation session of a total duration of T minutes is split amongst the stimulation units 210 in accordance with a predetermined protocol such that simulation is provided to different target sites (corresponding to the stimulation units 210) for varying durations and at different times (that is, not simultaneously). In various embodiment, T is within a range of 0-60 minutes and, more preferably, 30-60 minutes. In some embodiments, the sum of stimulation session durations split amongst different target sites is equal to the total duration of T minutes. It should be noted that, in some embodiments, it is not necessary that the stimulation session (of say T minutes) be split equally and amongst all the stimulation units 210. Thus, different stimulation units may deliver stimulation to corresponding target sites for different durations provided the total of all these durations is T minutes. Also, there may be one or more stimulation units which do not deliver stimulation at all. Therefore, in various embodiments, one or more stimulation units may deliver stimulation to corresponding target sites for a period of time ranging from 0-60 minutes. In various embodiments, a stimulation session may comprise a plurality of such asynchronous cycles of therapy. In some embodiments, stimulation is provided, within a stimulation session, for a period of time until a target limb or body portion motor function or position is corrected, rather than for a set time period (T minutes). For example, for a patient suffering from Parkinson's disease and having a shuffling gait, as further described with reference to FIGS. 5B and 5C, stimulation may be provided to a targeted limb until the patient takes footsteps that include a distance between each foot which is considered normal as determined by preset thresholds.

Referring now to FIG. 2B, in some embodiments, the head module 205 and the stimulation units 210 are triggered by the RRA, running on the remote management device 215, to stimulate the respective sites synchronously. In other words, a stimulation session is activated and coordinated by the RRA to deliver stimulation therapy simultaneously to the brain as well as to the targeted limbs. In some embodiments, the head module 205 and the stimulation units 210 stimulate the respective sites asynchronously. In other words, a stimulation session is activated and coordinated by the RRA such that the head module 205 and the stimulation units 210 do not deliver stimulation to the respective sites simultaneously. Thus, in some embodiments, for a stimulation session of a duration of M minutes—the head module 205 initiates stimulation of the brain for a predefined duration, such as X % of the total stimulation session duration of M minutes, while the stimulation units 210 remain inactive. Thereafter, the head module 205 stops delivering stimulation and the stimulation units 210 are triggered to deliver stimulation to the targeted limbs for a predefined duration, such as Y % of the total stimulation session duration of M minutes. In various embodiments, the X % and Y % durations may or may not be equal. Also, as discussed earlier with reference to FIG. 2A, the Y % of the total stimulation session duration of M minutes may be further split, equally or unequally, amongst some or all of the stimulation units 210 to be delivered to the corresponding target sites simultaneously or asynchronously. In some embodiments, M is in a range of 0-60 minutes and, more preferably, 30-60 minutes. In some embodiments, X % is in a range of 0-100% of the total stimulation session duration, Y % is in a range of 0-100% of the total stimulation session duration, and X %+Y % is equal to no more than 100% of the total stimulation session duration. An asynchronous cycle of stimulation therapy, in some embodiments, comprises of stimulating the brain (no stimulation of the targeted limbs), stopping stimulation of the brain, followed by stimulating the targeted limbs (synchronously or asynchronously) and then stopping stimulation of the targeted limbs, or vice-versa. In various embodiments, a stimulation session may comprise a plurality of such asynchronous cycles of therapy.

In accordance with aspects of the present specification, the coordinated synchronous and/or asynchronous stimulation of the brain and/or the targeted limbs (for example, during task related training sessions—TRT) improves the ability of the brain to control the targeted limbs by promoting neuron/synapse growth and development in the brain by taking advantage of its plasticity. Coordinated synchronous and/or asynchronous stimulation of the stimulation units alone or between the optional head module and the stimulation units encourages redevelopment of nerve connections between the brain and the targeted limb(s) or body region(s) controlled by that portion of the brain and addresses weakness and spasticity of the targeted limb(s) or body region(s) as well as gait and balance for patient rehabilitation. In some embodiments, after delivering stimulation therapy through at least ‘Z’ stimulation sessions or through a period of ‘W’ seconds, minutes, or hours, spasticity (measured on a composite spasticity scale) of the targeted limb or body portion improves by at least 5%. In some embodiments, stimulation is provided up to a threshold level, wherein the threshold level is defined at the level of stimulation where the patient becomes aware of the stimulation (for example, the patient experiences pain from the stimulation). In some embodiments, the number of stimulation sessions ‘Z’ is in a range of 1 to 24 per day. In some embodiments, the period ‘W’ is in a range of 1 second to 2 hours.

Referring to FIG. 2B, in accordance with some aspects of the present specification, only the stimulation units 210 are triggered to deliver stimulation while the optional head module 205 does not deliver stimulation to the brain. In accordance with still other aspects of the present specification, only the stimulation units 210 are placed on the user's body to deliver stimulation while no head module 205 is needed or placed at the user's head (as shown in FIG. 2A).

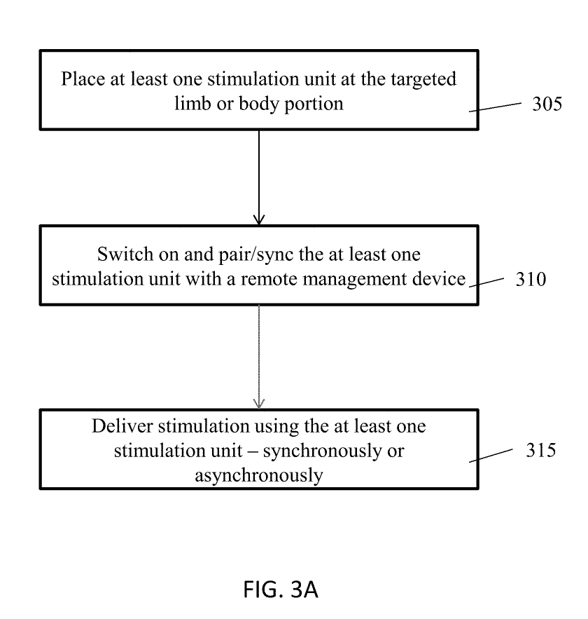

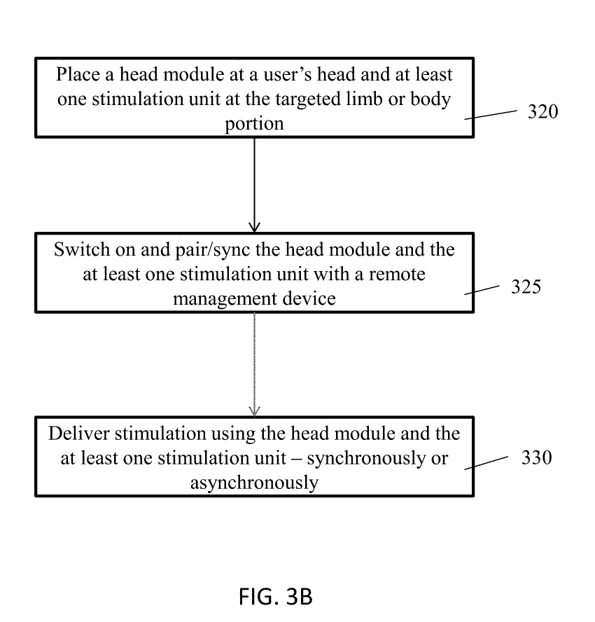

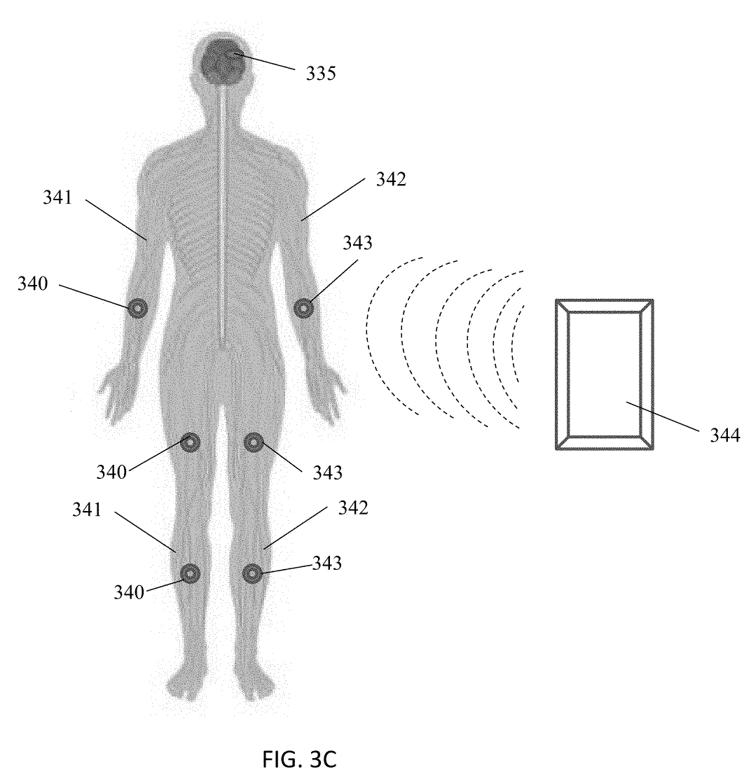

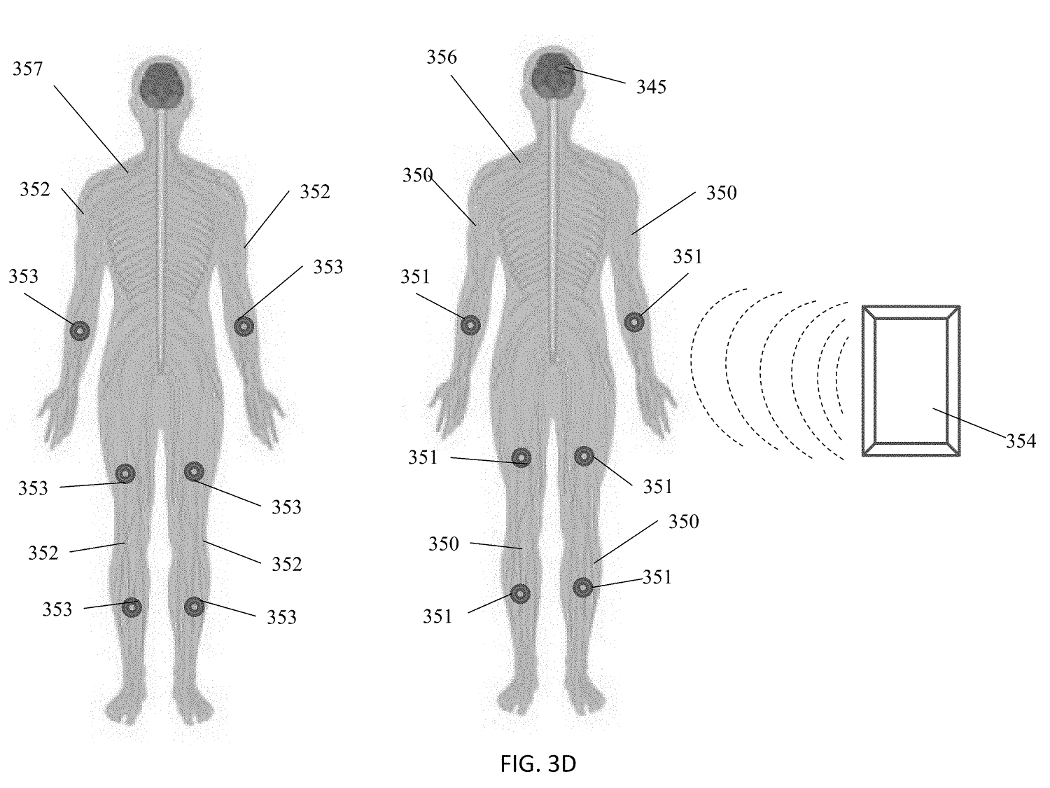

FIG. 3A is a flowchart illustrating a plurality of exemplary steps of a method of delivering stimulation to treat at least one targeted limb or body portion, using at least one stimulation unit, in accordance with some embodiments. At step 305, at least one stimulation unit is placed on a targeted limb or body portion of a user. The stimulation unit may be placed along any compromised limb or body portion. Thereafter, at step 310, the at least one stimulation unit is switched on and paired or synced with the user's smartphone (functioning as a remote management device) that runs the RRA software of the present specification. In embodiments, different types of remote management devices may be used that execute the RRA software exclusively or as a part of a system with other applications. Examples of remote management devices may include portable computers, laptops, PDAs, or other mobile processing devices.

At step 315, the RRA on the user's smartphone triggers the at least one stimulation unit to deliver stimulation to the targeted limb or body portion—synchronously or asynchronously, in accordance with various embodiments.

FIG. 3B is a flowchart illustrating a plurality of exemplary steps of a method of delivering stimulation to treat at least one targeted limb or body portion, using at least one head module along with at least one stimulation unit, in accordance with some embodiments. At step 320, a head module is placed on the user's head while at least one stimulation unit is placed on a targeted limb or body portion of the user. Thereafter, at step 325, the head module and the at least one stimulation unit are switched on and paired or synced with the user's smartphone (functioning as a remote management device) that runs the RRA software of the present specification. In other embodiments, different types of remote management devices may be used that execute the RRA software exclusively or as a part of a system with other applications. Examples of remote management devices may include portable computers, laptops, PDAs, or other mobile processing devices.

In various embodiments, a site of placement of the head module is dependent on the targeted limb or body portion. In one embodiment, the placement sites of the head module and of the at least one stimulation unit are contralateral. That is, if the targeted limb or body portion constitutes a left portion of the user's body then the head module is placed towards the right portion, area, or region of the user's head and vice versa. In some embodiments, where the targeted limbs or body portions constitute both left and right portions of the user's body a first head module is placed towards the right area of the user's head to coordinate, when needed, with at least one stimulation unit placed on the left targeted limb or body portion while a second head module is placed towards the left area of the user's head to coordinate, when needed, with at least one stimulation unit placed on the right targeted limb or body portion.

At step 330, the RRA on the user's smartphone triggers the head module and the at least one stimulation unit to deliver stimulation to the brain and the targeted limb or body portion—synchronously or asynchronously, in accordance with some embodiments. It should be appreciated that during an asynchronous stimulation therapy cycle the head module and the at least one stimulation unit do not deliver stimulation simultaneously. Also, in embodiments where a plurality of stimulation units are placed on the user's body, the plurality of stimulation units may deliver stimulation asynchronously amongst them as well as with reference to the head module. For example, in some embodiments, at least two stimulation units are placed on the user's body and the at least two stimulation units are activated asynchronously. In other embodiments, at least two stimulation units are placed on the user's body and the at least two stimulation units are activated synchronously.

Stimulation Parameters, Patterns and/or Protocols to Deliver Therapy

In various embodiments, stimulation patterns or protocols comprise driving, setting, customizing or adjusting a plurality of stimulation parameters such as, but not limited to, the number of stimulation sessions per day, duration of each stimulation, time or moment of application of the stimulation sessions, intensity of stimulations, stimulation pulse shape, frequency, width and amplitude, stimulation duty cycle, stimulation continuity profile, and minimum and maximum overall duration or course of stimulation treatment in days, weeks or months. Following are exemplary standard setting ranges for some of the stimulation parameters: Pulse Width: 10 μsec to 10 msec; Pulse Amplitude: 100 μA to 500 mA and any increment therein; Pulse Frequency: 1 Hz to 10,000 Hz, preferably 1 Hz to 100 Hz; Pulse Shape: Monophasic, biphasic, sinusoidal; Duty Cycle: 1% to 100%; Stimulation Session Duration: 1 min to 120 min or 50 ms to 120 min or substantially continuously; Number of Stimulation Sessions/Day: 1 to 24; Number of Sessions/Week: 1 to 168 or 1 to substantially continuously; and Electrode impedance (that is, the electrode-tissue interface impedance): 100 ohms to 5 kilo-ohms, 10 ohms to 5 kilo-ohms, 200 ohms to 1000 ohms, or 1 kilo-ohms to 100 kilo-ohms.

In embodiments, a duty cycle of stimulation is optimized or adjusted such that the stimulation units and the optional head unit remain in sleep mode between consecutive stimulation sessions that correspond to an active mode of the devices. In various embodiments, the sleep mode corresponds to an average current of less than 10 μA while the active mode corresponds to an average current range of 2 to 30 mA.

In some embodiments, the stimulation units (and the optional head module) provide electrical stimulation having the following parameters which are adjustable by the patient using the remote management device: Monophasic pulse shape with an active charge balancing phase; Pulse Width: 25 μsec to 400 μsec in steps of 25 μsec; Pulse Amplitude: 1 mA to 50 mA in steps of 1 mA; Pulse Frequency: from 1 Hz, 5 Hz, 10 Hz, 15 Hz, 20 Hz, 25 Hz, 30 Hz, 40 Hz, 50 Hz, 60 Hz, 70 Hz, 80 Hz, 90 Hz, 100 Hz, 150 Hz, 200 Hz; and Stimulation Session Duration: from 5 min to 60 min in steps of 5 min.

In some embodiments, the stimulation units (and the optional head module) provide electrical stimulation having the following parameters which are adjustable by the patient using the remote management device: Pulse Width: 100 μsec to 500 msec, preferably 10 μsec to 100 msec; Pulse Amplitude: 1 μA to 1 mA; Pulse Frequency: 0.1 Hz to 1 kHz; Stimulation Session Duration: 1 min to 24 hr; Number of Stimulation Sessions/Day: 1 to 24; and Number of Sessions/Week: 1 to substantially continuously.

In one embodiment, the electrical pulses, whether in a single waveform or multiple waveforms, only have a frequency of up to 200 Hz and no greater. In other words, the system does not deliver any electrical pulse that has a frequency in excess of 200 Hz. In another embodiment, the electrical pulses are delivered in a single waveform and do not take the form of multiple waveforms integrated or combined together.

### Posture Restoration, Feedback and Training

In accordance with some aspects of the present specification, one or more stimulation units on standalone basis or in tandem with an optional head module, and in data communication with the remote management device (running the Rehabilitation and Restoration software application—RRA), enable detection of a posture, position, movement or gait of a user's targeted limb or body portion and trigger a stimulation session and/or a feedback, which may be tactical, audible and/or visual, to signal to the user when his targeted limb or body portion is not in an appropriate position, movement, gait or posture. It should be appreciated that the targeted limb or body portion may or may not be affected, diseased or dysfunctional in various embodiments.

In accordance with some embodiments, the user's targeted limb or body portion is kept in an optimal or desired position, posture, gait or movement either by the user or under the assistance or guidance of a medical professional, physiotherapist, coach or trainer. In some embodiments, at least one stimulation unit is then positioned on the targeted limb or body portion while the optional head module is positioned on the user's head. It should be appreciated that in some embodiments, the head module is not positioned on the user's head and only at least one stimulation unit is positioned and used on the targeted limb or body portion. Thereafter, actuating a button on the at least one stimulation unit (or the at least one stimulation unit and the head module, in accordance with some embodiments), or actuating a GUI based icon or button on the user's smartphone (functioning as the remote management device) causes the RRA to acquire and record the current optimal or desired position, posture, gait or movement of the targeted limb or body portion. Subsequently, any change or deviation of the targeted limb or body portion, from the recorded optimal or desired position, posture, gait or movement (also referred to as a ‘reference model’ or ‘reference map’) is continuously monitored and recorded by the RRA. Additionally, the recorded deviation of the targeted limb or body position, posture, gait or movement results in at least one of the following actions: triggering stimulation by the at least one stimulation unit (or by the at least one stimulation unit and the head module, in accordance with some embodiments); and triggering an audio, visual and/or tactile alarm or feedback (for example, vibratory). In various embodiments, aggregation of the monitored change or deviation of the targeted limb or body portion, over a period of time, enables generation of a positional, behavioral or movement map (also referred to as an ‘impaired model’ or ‘impaired map’) indicating a behavior of the targeted limb or body portion for study by a therapist or coach.

Therefore, in various embodiments, a ‘reference model’ or ‘reference map’ is defined as a baseline position of a limb or body portion or baseline movement of a limb or body portion. In various embodiments, the ‘reference model’ or ‘reference map’ can be obtained from: a target limb or body portion of a patient, which may or may not be affected, diseased or dysfunctional; a limb or body portion of the patient which is not targeted (for example, an unaffected right leg of a patient when a patient has an affected left leg); and/or a limb or body portion of a third person (not the patient). Deviations in position or movement of the target limb or body portion from the ‘reference model’ or ‘reference map’ are analyzed by the system and stimulation and/or feedback are provided. An ‘impaired model’ or ‘impaired map’ is defined as an aggregation of deviations from the ‘reference model’ or ‘reference map’ over time.

In some embodiments, the alarm or feedback along with the positional, behavioral or movement map (the ‘reference model’ or ‘reference map’) of the targeted limb or body portion is utilized for persons such as, but not limited to, joggers, horseback riders, gymnasts, and dancers for whom correct posture, gait or bodily movement or coordination is vital. For such persons, in some embodiments, optionally frontal lobe stimulation (through the head module) of the brain may encourage fast learning and heightened awareness of correct body posture and motion pattern.

In some embodiments, referring to FIG. 3C, the ‘reference model’ or ‘reference map’ is generated by placing one or more stimulation units 343 on an unaffected or healthy limb or body portion 342 corresponding to an affected or dysfunctional limb or body portion of a user and thereafter monitoring and recording the user's body parameters such as position, posture, gait or movement. For example, consider a case of a patient whose limbs or body portions 341 on one side, say the right side, have been affected due to a stroke or trauma while the corresponding limbs or body portions 342 on the other side, the left side, are unaffected or healthy in terms of body parameters such as posture/movements/gait. In another example, limbs or body portions of a patient, on one side of his body (say, the right side), may be affected by Parkinsonian gait whereas the corresponding limbs or body portion of the patient, on the other side (say, the left side) of the body, may be unaffected and healthy. In such cases, the system is used to generate a ‘reference model’ or ‘reference map’ of the unaffected or healthy limbs or body portions 342 (on the left side, in these examples). The affected or dysfunctional limbs or body portions 341 (on the right side, in these examples) are then monitored, compared, treated and corrected (through stimulation of the affected or dysfunctional limbs or body portions and/or real-time feedback and training, for example) via stimulation units 340, optional head module 335 and remote management device 344, with reference to the ‘reference model’ or ‘reference map’. Stimulation units 340, stimulation units 343 and optional head module 335 are all in wireless communication with the remote management device 354. In other words, the ‘reference model’ or ‘reference map’ is generated from the healthy limbs or body portions of the same user who's corresponding other limbs or body portions are affected or dysfunctional.

In some embodiments, referring to FIG. 3D, the ‘reference model’ or ‘reference map’ is generated by placing one or more stimulation units 353 on unaffected or healthy limbs or body portions 352 of a healthy “reference” person or third party 357 (different from the patient 356 whose limbs and/or body portion 350 are affected). For example, consider a case of a patient 356 whose limbs or body portions 350 on both sides of the body have been affected due to stroke or trauma or may be affected by Parkinsonian gait. In such cases, the system is used to generate a ‘reference model’ or ‘reference map’ of the unaffected or healthy limbs or body portions 352 of the healthy “reference” person 357 via stimulation units 353 and a remote management device 354. The thus generated ‘reference model’ or ‘reference map’ is then used to monitor, compare, treat and correct the posture/movements/gait of the affected or dysfunctional limbs or body portions 350 of the patient 356 using stimulation units 351, optional head module 345 and remote management device 354. Stimulation units 353, stimulation units 351 and optional head module 345 are all in wireless communication with the remote management device 354. In some embodiments, the third party is a healthy individual whose relevant limbs or body portions exhibit healthy/normal posture, position, gait or movement. In some embodiments, the third party is an athlete, sportsperson, dancer, model, acrobat, gymnast or any other performer. The ‘reference model’ or ‘reference map’ generated with such a third party may be used as a ‘standard’ for feedback, coaching and training of other users aspiring to improve their performance in fields of activities similar to the third party. In some embodiments, the third party or healthy “reference” person may be selected so that they match the physical characteristics or attributes of the patient (such as but not limited to height and weight). In some embodiments, a reference library of ‘reference models’ or ‘reference maps’ may be created so that the system can select at least one reference. In some embodiments, the system may select portions of reference attributes from a reference library to arrive at a ‘reference model’ or ‘reference map’.

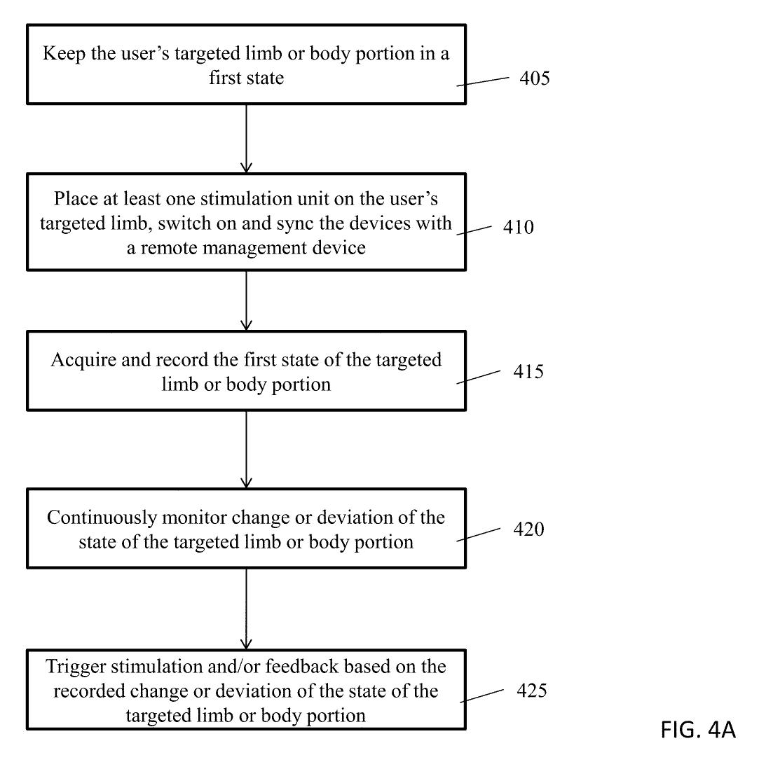

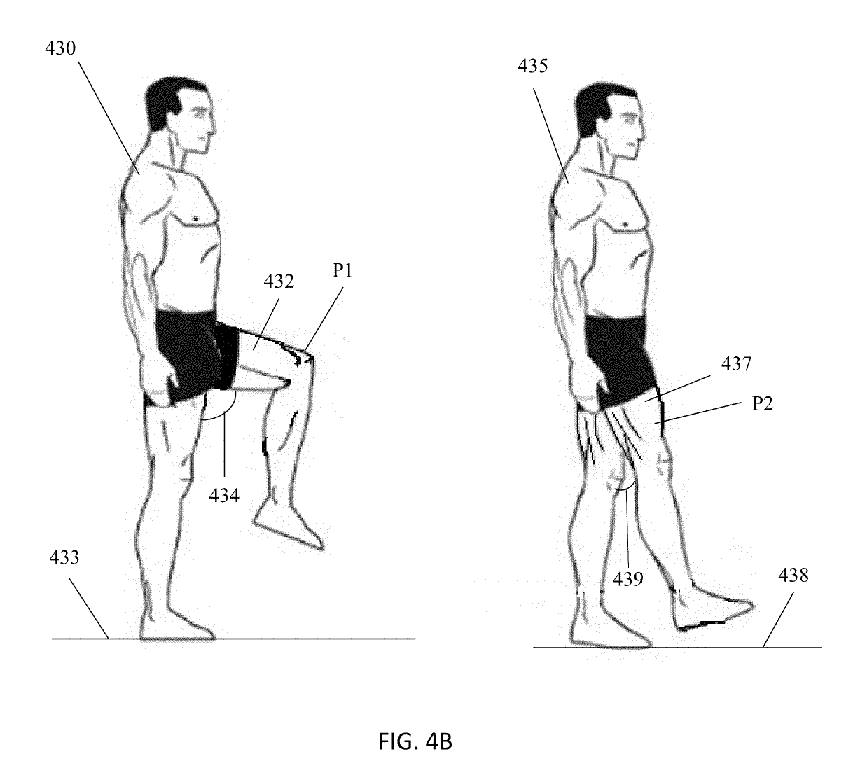

FIG. 4A is a flowchart illustrating a plurality of exemplary steps of a method of monitoring and recording a movement, position, posture, bearing or gait of a user's targeted limb or body portion that may or may not be affected, dysfunctional, diseased or errant. At step 405, the user's targeted limb or body portion is kept in a first state. The first state corresponds to a desired, optimal or correct position, posture, gait or movement of the targeted limb or body portion and therefore constitutes a ‘reference model’ or ‘reference map’ for the user. In one embodiment, the user is someone who has suffered a stroke or trauma that has affected his right leg, for example. Referring to FIG. 4B, a healthy individual 430 can lift his leg so that his thigh 432 is at position P1, at least parallel to the ground plane 433 (roughly a right angle with reference to the other leg) or at an obtuse angle 434 with reference to the other leg. Angle 434 defines a ‘reference model’, ‘reference map’, or baseline position for the system when the thigh 432 is at position P1. The user 435 who is recovering from a stroke is unable to get his thigh 437 so that it is parallel to the ground 438 (that is, may not be able to get his hip and knee parallel to the ground). Typically, the user is able to, without aid, get his thigh 437 to position P2, to make just an acute angle 439 to the other leg. The difference between angle 439 and angle 434 is the deviation of the position of the targeted limb or body portion from the baseline position and is used to trigger stimulation or feedback if the deviation exceeds a predefined deviation threshold. Thus, in this embodiment, the first state corresponds to the user keeping his right thigh at least parallel to the ground—with the help of a medical practitioner or by himself using his hands to lift his thigh up enough to be at least parallel to the ground, for example.

In another example, the user may be a healthy person who is training or exercising using a specific jogging technique wherein the user jogs standing at a position, without any translational movement, and wherein every jogging movement requires the user to lift his legs such that their thighs are almost parallel to the ground. In various embodiments of the present specification, a healthy person is defined as an individual with muscles that do not have an impaired motor function.

Referring again to FIG. 4A, at step 410, at least one stimulation unit is placed on the targeted limb or body portion. Optionally, a head module may also be placed on the user's head. For example, in the case of the user recovering from stroke, stimulation units may be positioned on the user's thigh, lower leg and the foot to be able to determine angles along the corresponding body portions and generate information related to whether the thigh is raised at least parallel to the ground. Thereafter, the optional head module and the at least one stimulation unit are switched on and paired or synced with the user's smartphone (functioning as a remote management device) that runs the RRA software of the present specification. It should be noted that the sequence of steps 405, 410 are interchangeable in various embodiments.

Also, in embodiments where a head module is also used in tandem with one or more stimulation units, a site of placement of the head module is dependent on the targeted limb or body portion. In one embodiment, the placement sites of the head module and of the at least one stimulation unit are contralateral. That is, if the targeted limb or body portion constitutes a left portion of the user's body then the head module is placed towards the right portion, area or region of the user's head and vice versa. In some embodiments, where the targeted limbs or body portions constitute both left and right portions of the user's body a first head module is placed towards the right area of the user's head to coordinate, when needed, with at least one stimulation unit placed on the left targeted limb or body portion while a second head module is placed towards the left area of the user's head to coordinate, when needed, with at least one stimulation unit placed on the right targeted limb or body portion.

At step 415, a button on the at least one stimulation unit (and on the head module, when also used in an optional embodiment), or a GUI based icon or button on the smartphone (functioning as the remote management device) is actuated to trigger the RRA to acquire and record the first state of the targeted limb or body portion. Thereafter at step 420, the RRA continuously monitors any movement, change or deviation of the state of the targeted limb or body portion with reference to the earlier recorded first state or ‘reference model’ or ‘reference map’. For example, during a physiotherapy session the user may be required to raise his right leg and try to get his thigh at least parallel to the ground. The RRA continuously monitors any change or deviation of the position of the right thigh, during the physiotherapy session, in comparison to the earlier recorded ‘reference model’ or ‘reference map’. The movement, change or deviation of the targeted limb or body portion is monitored by acquiring, processing and analyzing a plurality of signals generated by the accelerometer or inclinometer incorporated within the at least one stimulation unit. It should be appreciated that at steps 415 and/or 420, the at least one stimulation unit may be operated alone on a standalone basis or in tandem with the head module. Accordingly, in embodiments, use or placement of the head module may not be needed and only the at least one stimulation unit is positioned on the targeted limb or body portion.

At step 425, the recorded deviation of the targeted limb or body position results in at least one of the following actions: triggering stimulation at the at least one stimulation unit and/or at the head module; and triggering an audio, visual and/or tactile alarm or feedback (for example, vibratory). In some embodiments, the at least one of the aforementioned actions are triggered only if the deviation of the position of the targeted limb or body portion is beyond a predefined deviation threshold with reference to the ‘reference model’ or ‘reference map’.

In various embodiments, aggregation of the monitored movement, change or deviation of the targeted limb or body portion, over a period of time, enables generation of a positional or movement map indicating a behavior of the targeted limb or body portion for study by the medical professional or coach. In some embodiments, the alarm or feedback along with the positional, behavioral or movement map of the targeted limb or body portion is utilized for persons such as, but not limited to, joggers, horseback riders, gymnasts, and dancers for whom correct posture, gait or bodily movement or coordination is vital. For such persons, a ‘reference model’ or ‘reference map’ may be generated by these persons themselves performing the activities in ideal or optimal fashion and in controlled environments or may be generated using other individuals who may be experts, specialists or optimally trained to perform the activities.

In accordance with alternative embodiments, to monitor and identify errant behavior in a dysfunctional or affected limb or body portion (targeted′ limb or body portion) and to resultantly deliver stimulation therapy, at least one first stimulation unit is positioned on the targeted limb or body portion while at least one second stimulation unit is positioned on a corresponding healthy limb or body portion. The at least one second stimulation unit, by virtue of the signals generated by the accelerometer or inclinometer included therein, functions as an optimal or correct movement, positional, posture or gait reference (reference model′ or ‘reference map’) for the at least one first stimulation unit. In some embodiments, an optional head module is also placed at the user's head for coordination with the first and second stimulation units. However, in some alternative embodiments, the head module is not required and the first and second stimulation units function on a stand-alone basis.

FIG. 5A is a flowchart illustrating a plurality of exemplary steps of a method of monitoring and recording a position, posture, bearing, behavior or gait of a user's targeted limb or body portion that, in some embodiments, may be affected, dysfunctional, diseased or errant. At step 505, at least one first stimulation unit is placed on the user's targeted limb or body portion and at least one second stimulation unit is placed on the user's healthy limb or body portion corresponding to the targeted limb or body portion. In some embodiments, a head module may also be optionally placed on the user's head. For example, if the targeted limb of the user is a left arm then the corresponding healthy limb is the user's right arm and vice versa. This may happen in patients suffering from stroke, trauma or other neurological conditions such as Parkinsonian gait or any other condition affecting motor functioning and coordination. Thereafter, the first and second stimulation units (and the optional head module, if used in some embodiments) are switched on and paired or synced with the user's smartphone (functioning as a remote management device) that runs the RRA software of the present specification.

At step 510, the at least one second stimulation unit generates a plurality of reference signals corresponding to the movement, position, posture, bearing or gait of the healthy limb or body portion. These reference signals constitute a ‘reference model’ or ‘reference map’ for the user. Similarly, the at least one first stimulation unit generates a plurality of target signals corresponding to the movement, position, posture, bearing or gait of the affected limb or body portion. These target signals constitute an ‘impaired model’ or ‘impaired map’. At step 515, the RRA continuously acquires, compares and analyzes the ‘impaired model’ or ‘impaired map’ in relation to the ‘reference model’ or ‘reference map’ in order to monitor change or deviation of the targeted limb or body portion with reference to the corresponding healthy limb or body portion. In various embodiments, the monitored change or deviation is targeted towards identifying abnormal or errant behavior of the targeted limb or body portion.

In embodiments where a head module is also used in tandem with one or more stimulation units, a site of placement of the head module is dependent on the targeted limb or body portion. In one embodiment, the placement sites of the head module and of the at least one first stimulation unit are contralateral. That is, if the targeted limb or body portion constitutes a left portion of the user's body then the head module is placed towards the right portion, area or region of the user's head and vice versa. Also, in alternate embodiments, at steps 505, 515 the first and second stimulation units may be operated alone on a standalone basis or in tandem with the head module. Accordingly, in some embodiments use or placement of the head module may not be needed and only the first and second stimulation units are positioned on the targeted and healthy limbs or body portions, respectively.

At step 520, the recorded deviation of the ‘impaired model’ or ‘impaired map’ corresponding to the targeted limb or body portion in comparison to the ‘reference model’ or ‘reference map’ corresponding to the healthy limb or body portion results in at least one of the following actions: triggering stimulation at the at least one first stimulation unit and/or at the head module (in embodiments where the head module is used in tandem with the first and second stimulation units); and triggering an audio, visual and/or tactile alarm or feedback (for example, vibratory). In some embodiments, the at least one of the aforementioned actions are triggered only if the deviation of the targeted limb or body portion is beyond a predefined deviation threshold with reference to the corresponding healthy limb or body portion (that is, the ‘reference model’ or ‘reference map’).

In various embodiments, aggregation of the monitored movement, change or deviation of the targeted limb or body portion, over a period of time, enables generation of a positional, behavioral or movement map (the ‘impaired model’ or ‘impaired map’) indicating a behavior of the targeted limb or body portion for study by a medical professional or coach.

FIG. 5B is another flowchart illustrating exemplary steps of a method of delivering stimulation to provide therapy and/or real-time feedback to a patient with discontinuous movement disorder or motor deficit such as those associated with Parkinsonian gait, in accordance with some embodiments. In alternative embodiments, the method described in context of FIG. 5B may be applicable to any patient with mobility disorders, dysfunction or deficit (that can be remedied with therapy) such as, but not limited to, subcortical arteriosclerotic encephalopathy (SAE) or cerebellar ataxia.

At step 525, a stimulation unit is placed at regions on a dysfunctional targeted limb or body portion of the patient which are capable of controlling one or more motor functions of the patient related to the dysfunctional targeted limb or body portion. In an embodiment, a plurality of stimulation units are placed at various regions, related to the dysfunctional targeted limb or portion, of the body controlling one or more motor functions such as those related to walking, for example. In embodiments, the regions may include an area under a big toe, near an ankle, near a knee, lower leg, upper leg, arm or any other joint that may impact the patient's mobility. Optionally, a head module may also be placed on the user's head.

In some embodiments, the patient may already be receiving therapy, such as deep brain stimulation (DBS), involving implantation of a neurostimulator (also referred to as a ‘brain pacemaker’) that sends electrical impulses, through implanted electrodes, to specific targets in the brain (brain nuclei) for the treatment of movement and neuropsychiatric disorders. In such embodiments, the plurality of stimulation units along with the remote management device and the RRA executed thereon, complement the DBS treatment by coordinating stimulation therapy with the implanted neurostimulator and/or titrating stimulation therapy delivered by the plurality of stimulation units considering the DBS treatment.

Thereafter, at step 527 the stimulation units are switched on and paired or synced with the patient's remote management device. The remote management device may be a smartphone, or any other device configured to remotely communicate with the stimulation units, and also configured to run the RRA software of the present specification.

At step 529 the plurality of stimulation units generate target signals corresponding to the movement, position, posture, bearing or gait of the patient during activities such as, but not limited to, walking, getting up from a sitting position, sitting from a standing position or getting up from a lying down position. These target signals constitute an ‘impaired model’ or ‘impaired map’.

The ‘impaired model’ or ‘impaired map’ of the patient is characterized by small shuffling steps, a general slowness of movement, reduced stride length and walking speed during free ambulation but increased double support duration and cadence rate. For example, referring to FIG. 5C, in an embodiment, an individual with a normal gait takes footsteps wherein a first foot 535 is separated from a second foot 537 by a distance ‘D’, wherein distance ‘D’ defines the ‘reference model’, ‘reference map’, or baseline distance between feet 535, 537 for a normal footstep. An individual suffering from Parkinson's disease might make shuffling footsteps, wherein a first foot 538 is separated from a second foot 540 by a distance ‘d’, wherein distance ‘d’ is shorter than distance ‘D’. Distance ‘d’ is compared by the system to distance ‘D’, the baseline or ‘reference model’ or ‘reference map’ to determine a deviation from baseline. If the deviation exceeds a predefined threshold deviation, the system triggers stimulation or feedback to assist the patient in improving the motor function. In some embodiments, stimulation units are positioned on normal feet 535, 537 and stimulation units are positioned on impaired feet 538, 540. Over time, the differences in distances between each foot 535, 537 in the normal set and distances between each foot 538, 540 in the impaired set, during walking, are compared to create ‘impaired models’ or ‘impaired maps’. In various embodiments, the ‘impaired model’ or ‘impaired map’ of the patient exhibits gait characteristics such as, but not limited to the following:

a) Heel to Toe Characteristics. While in normal gait, the heel strikes the ground before the toes (also called heel-to-toe walking), whereas in Parkinsonian gait, motion is characterized by flat foot strike (where the entire foot is placed on the ground at the same time) or less often and in the more advanced stages of the disease by toe-to-heel walking (where the toes touch the ground before the heel). In addition, Parkinson patients have reduced foot lifting during the swing phase of gait, which produces smaller clearance between the toes and the ground. Also, Parkinson patients show a trend towards higher relative loads in the forefoot regions combined with a load shift towards medial foot areas. This load shift is believed to help in compensating for postural imbalance.

b) Vertical Ground Reaction Force (GRF). In normal gait, the vertical ground reaction force (GRF) plot has two peaks—a first peak, which can be defined as when the foot strikes the ground and a second peak, which is caused by push-off force from the ground. The shape of the vertical GRF signal is abnormal in Parkinson patients. In the earlier stages of the disease, reduced forces (or peak heights) are found for heel contact and the push-off phase resembling that of elderly subjects. In the more advanced stages of the disorder where gait is characterized by small shuffling steps, Parkinson patients show only one narrow peak in the vertical GRF signal.

c) Falls and Freezing of Gait. Falls and freezing of gait are two episodic phenomena that are common in Parkinsonian gait. Freezing of Gait (FOG) is typically a transient episode, lasting less than a minute, in which gait is halted and the patient complains that his/her feet are glued to the ground. When the patient overcomes the block, walking can be performed relatively smoothly. Falls are mainly a result of sudden changes in posture, such as turning movements of the trunk, or a result of attempts to perform more than one activity simultaneously with walking or balancing.

d) Postural Sway. Postural instability in an upright stance compromises the ability to maintain balance during everyday tasks such as walking, turning and standing up from sitting.

In embodiments, a ‘reference model’ or ‘reference map’ corresponding to a normal gait is used to compare the patient's abnormal gait, also referred to as the ‘impaired model’ or ‘impaired map’. In accordance with some aspects, the ‘reference model’ or ‘reference map’ is preferably generated by placing a plurality of reference stimulation units at various regions of the body, related to a healthy or normally functioning limb or portion and corresponding to the dysfunctional limb or portion, controlling one or more motor functions such as those related to walking, for example. In other words, it is preferable to generate the ‘reference model’ or ‘reference map’ by acquiring reference signals from the reference stimulation units placed on the healthy limb or body portion of the same patient. However, this is possible only in cases where the patient is suffering from motor dysfunction, such as Parkinsonian gait, on certain portions of his body only while corresponding other portions of his body are healthy and functioning normally to be utilized for generating the reference signals.

In accordance with some aspects, where the patient is suffering from motor dysfunction and it is not possible to generate reference signals from other corresponding portions of his body, the ‘reference model’ or ‘reference map’ used is that of a third person (other than the patient) who is healthy and performs related activities, such as walking for example, normally. In embodiments, such a ‘reference model’ or ‘reference map’ may be pre-stored at a central server from where it can be accessed by the remote management device, executing the RRA of the present specification, associated with the plurality of stimulation units placed on the body of the patient.

At step 531, the RRA continuously acquires, compares and analyzes the ‘impaired model’ or ‘impaired map’ in relation to the ‘reference signals model or map’. At step 533, the recorded deviation of the ‘impaired model’ or ‘impaired map’ corresponding to the targeted limb or body portion in comparison to the ‘reference model’ or ‘reference map’ results in at least one of the following actions: triggering stimulation at one or more of the plurality of stimulation units and/or at the head module (in embodiments where the head module is used in tandem with the stimulation units); and triggering an audio, visual and/or tactile alarm or feedback (for example, vibratory). In some embodiments, the at least one of the aforementioned actions are triggered only if the deviation of the targeted limb or body portion is beyond a predefined deviation threshold with reference to the ‘reference model’ or ‘reference map’. In some embodiments, stimulation at one or more of the plurality of stimulation units and or at the head module (in embodiments where the head module is used in tandem with the stimulation units) and/or an audio, visual and/or tactile alarm or feedback is provided for a duration of T minutes. In various embodiments, T is within a range of 0-60 minutes and, more preferably 30-60 minutes.

Optionally, in other embodiments, at step 534, the system continues to monitor and compare the ‘impaired model or map’ with the ‘reference model or map’ while stimulation and/or alarm or feedback are being provided. Once motor function has been corrected, at step 535, the system switches off stimulation and/or alarm feedback. In various embodiments, motor function is considered corrected when the ‘impaired model’, ‘impaired map’ or deviation of the targeted limb or body portion falls back below the predefined deviation threshold with reference to the ‘reference model’ or ‘reference map’, as acquired, compared, and analyzed by the RRA.

In various embodiments, aggregation of the monitored movement, change or deviation of the targeted limb or body portion, over a period of time, enables generation of a positional, behavioral or movement map (the ‘impaired model’ or ‘impaired map’) indicating a behavior of the targeted limb or body portion for study by a medical professional or coach.

In an embodiment, the present specification describes a novel system and method to provide real time feedback and/or coaching to patients such as the stroke recovery patients, patients with Parkinsonian gait, and other users such as athletes, for improving their normal or performance parameters such as the posture or movement or gait of a specific limb or the body portion.

In an embodiment, a plurality of positional sensors such as but not limited to micro sensors including accelerometers, inclinometers, gyroscopes, and GPS sensors are positioned on various limbs or body parts which needs to be monitored. These positional sensors capture various body parameters such as but not limited to the position or posture, movement, gait of a user and transmits this information to a control unit (such as the remote management device 115 of FIG. 1A, 1B) which evaluates such information and provides real time feedback to the user in case any of the body parameters deviates from an expected or reference range. In various embodiments, ‘real time’ is defined as less than one day, 12 hours, 6 hours, 1 hour, 10 minutes, 1 second, or any increment therein. In an embodiment, the system provides real time information to the user through visual/audio/vibration alarms. In another embodiment, the system provides real time information in the form of a dashboard in a hand held device such as the mobile phone or tablet. The user can track such information and in case of any deviation from the reference range of such parameters, the user can take corrective actions. In an embodiment, the corrective actions comprise modifying the movement and/or posture of a limb to bring the parameters related to such limb in the reference range. In another embodiment, the information on body parameters captured by various positional sensors is transmitted to a healthcare professional such as the physician to evaluate the results and recommend corrective action if required. For example, in case of a patient such as the stroke patient, the physician can regularly monitor the performance of the affected limbs or body parts against an expected or reference level and in case required, he can modify the therapeutic treatment being provided to the patient. In some embodiments, the sensors described above are purely informational, providing posture/movement information without providing stimulation. In other embodiments, the sensors are configured in the form of stimulation units 110 of FIG. 1A, 1B that not only monitor and provide posture/movement information but also provide stimulation therapy when needed. In various embodiments, one or more positional sensors as well as one or more stimulation units 110 of FIGS. 1A, 1B may be used in combination to monitor and provide posture/movement information and also provide stimulation therapy when needed.

In an embodiment, the system comprises a control unit including a computer program having an algorithm which can analyze the parameters captured by various positional sensors and can provide real time coaching or feedback. In various embodiments, the system comprises a display wherein the control unit is adapted to receive, and transmit to the display, a plurality of media and wherein said media provides real time coaching or feedback in a form of suggestions or recommendations intended to improve at least one motor function of a patient. In an embodiment, information is gathered and stored over time to provide a progressive record (in the form of a progress report, for example) to be compared with other athletes or patients with similar conditions. For example, in case of a stroke patient, the computer program analyzes the position and movement of the affected limbs in real time and immediately informs the patient if any deviation from an expected behavior is detected. Such real time feedback expedites the recovery process.

In various embodiments, the system comprises a controlling device in communication with a plurality of sensors configured to measure motion/gait/posture against a pre-defined normal or standard (“gold standard”) and advises the user accordingly. In some embodiments, the sensors comprise multiple wearable, smart phone controlled sensors. In embodiments, the sensors are worn bilaterally and include clip-on sensors, stick-on sensors, and/or bracelets. In some embodiments, the norm or standard comprises values obtained from the other side of the body where symmetry counts and/or from a prior performance of the activity which was deemed satisfactory and worthy of repetition. For example, in an embodiment, for a stroke patient or a patient who has had hip replacement surgery, the standard is determined by the unimpaired side of the body, either in real-time or from a prior recorded posture movement which was deemed satisfactory. In another embodiment, for athletic performance such as swimming or running, the standard is determined by comparing bilateral symmetry. The smart phone, or similar controlling device, processes the information received from the plurality of sensors. The smart phone or controlling device uses an algorithm to aggregate information from the multiple sensors and provide feedback to the user. In embodiments, the feedback is based on real-time comparisons and/or comparisons to recorded historic performance. In some embodiments, the feedback comprises auditory notifications to the user. In some embodiments, historic performance charts are generated by the system, based on recorded patient or user data, for comparison with performance data from healthy patients or well-known athletes to illustrate differences.

In various embodiments of the present specification, the parameters such as the posture/movement/gait of a targeted limb or body portion are compared to the reference parameters to understand and correct the abnormality or deviation. In an embodiment, the reference parameters are estimated by sampling similar parameters for a set of people belonging to same age group. However, in several cases, using the reference parameters estimated from set of people belonging to same age group may not be an accurate method as each individual has different characteristics. Therefore, in some embodiments of the present specification, the method to estimate the reference parameters used in a therapy depends on the specific conditions of the subject or patient and hence may vary in each case. In an exemplary embodiment comprising providing rehabilitation therapy to a stroke patient with a paralysis in one of his leg, the reference posture/movement/gait is estimated from the corresponding posture/movement/gait of his other unaffected leg by using bilateral positional sensors positioned on both the legs. Deriving the reference parameters from the corresponding unaffected limb or body portion is very accurate as it automatically takes specific condition of the user such as his age, agility level and other medical and fitness conditions into consideration. In an embodiment, information related to the deviation is quantified and the patient is provided with the exact level/degree of deviation which helps the patient to understand the problem in deep detail and accordingly take corrective actions.

In an embodiment of the present specification, the reference range of parameters is estimated from the best possible value for such parameters which is then treated as a gold standard. In an embodiment, the sportsmen such as athletes or swimmers can use the systems and methods of the present specification to enhance their performance level by correcting their posture or movement or gait. Positional sensors positioned on the targeted limbs or body portions provide feedback to a control unit which compares the captured body parameters with the gold standard for such parameters. In an embodiment, the gold standard for such parameters is derived from the similar parameters of best performing individuals in a field. For example in case of athletics or swimming, the reference parameters are estimated by recording the similar information for best athletes or swimmers in the world.

In another embodiment, the reference parameters are estimated by making the user stimulate the activity under a control environment in which the user can position or move his limb or body portion in the desired manner. The positional sensors record such position or movement or gait and store the corresponding information as a reference value. The ability of this system to record such correct posture or movement (at the click of a button) and use that as a reference point against which the future performance is measured is very advantageous. For example a runner or horseback rider might be coached to use perfect form while wearing the system and the system would then use that recording as a “gold standard” against which the future performance of the individual is evaluated. The system captures the body parameters of a user under subsequent random or normal conditions and compares it with the reference values derived under the control environment. The user is provided real time information on how his actual parameters stand with respect to the reference values.

In an embodiment, the user can select the reference parameters from a list of available reference parameters for specific activities. For e.g. an athlete may want to develop the stance and action of one of the renowned athletes such as Usain Bolt or Carl Lewis. In an embodiment, the system of the present specification comprises the reference range of parameters corresponding to a list of renowned athletes such as Usain Bolt or Carl Lewis and the user can select the reference range corresponding sporting personality that he would like to copy. The system subsequently compares the body parameters of the user with that of the reference parameters available for the selected sporting personality to provide real time feedback to the user showing his standing with respect to the reference level. In embodiments, the system also provides coaching to the athlete to improve his performance.

In another embodiment, the system comprises a list of gold standard or reference parameters based on the type of sporting activity. For example in case a swimmer wants to improve his butterfly swimming technique, he can select the standard or reference value for the butterfly swimming technique. Usually these reference parameters are derived from a sample of people who have excelled in the similar field.

Currently, there are insufficient data driven systems to understand if some therapy is leading to the right results for a patient. The patient does not know how much recovery should be expected after undergoing a therapy for a specific period of time. For example a stroke patient with a paralytic right arm does not know the ideal improvement should be expected after two months of physiotherapy sessions and/or electrical stimulating sessions. In an embodiment, the system of the present specification stores the information related to body parameters of a user over time to provide a progressive record (in the form of a progress report, for example) of recovery or development which could be compared with that of other patients or athletes having similar conditions. For e.g. in an embodiment the system can compare the percentage recovery of a stroke patient with that of other patients with similar condition who used the same data system in the past. In an embodiment the system develops artificial intelligence over time through a self-learning mechanism.

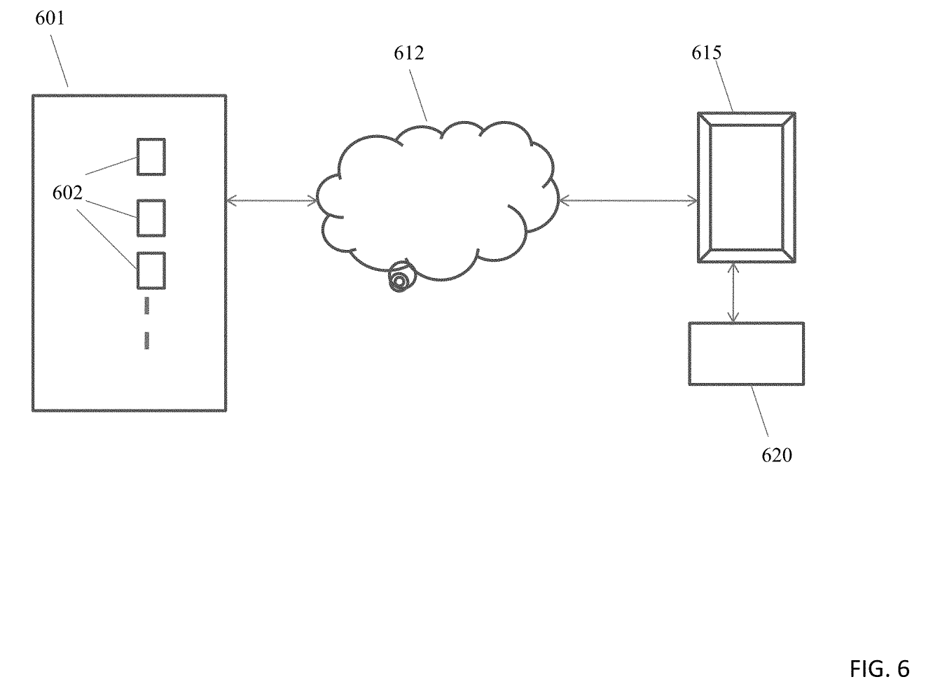

FIG. 6 illustrates a system 600 for performance feedback and coaching in accordance with an embodiment of the present specification. As shown in FIG. 6, the system 600 comprises a wearable unit 601 and a remote management device 615. The wearable unit 601 comprises a plurality of sensors 602. In an embodiment, the user can wear the wearable unit 601 such that the sensors 602 establish a contact with the targeted limbs or body portions which have to be monitored. In an embodiment, sensors 602 comprise any type of sensors which can record body parameters. In an embodiment, the sensors 602 comprise one or more accelerometer, inclinometer and gyroscope. In some embodiments, the sensors 602 also comprise a blood pressure monitor and/or heart rate monitor and/or a pulse monitor. It should be appreciated that, in various embodiments, the wearable unit 601 and the corresponding sensors 602 are worn or positioned using means such as adhesive, belt or strap. In some embodiments, the targeted limb or body portion may be weak, paralyzed, errant, diseased or dysfunctional due to neurological stroke or trauma or a motor dysfunction such as Parkinsonian gait, for example. In some embodiments, the targeted limb or body portion may be healthy or normal yet requiring behavioral, postural or movement related monitoring, feedback and training.

In various embodiments, the wearable unit 601 and the remote management device 615 are in data communication with each other using any one or a combination of cellular, Internet, TCP/IP, Ethernet, Bluetooth, wired, or wireless network 612. In a preferred embodiment, the wearable unit 601 and the remote management device 615 are in wireless data communication with each other. In an embodiment, the various positional sensors 602 capture the body parameters such as posture/movements/gait of the corresponding limb or body portion and transmit this information to the remote management device 615. In an embodiment, each of the sensors 602 can directly communicate with the remote management device 615. In another embodiment, the wearable unit 601 comprises a central controller which controls the various sensors 602 and the sensors 602 communicate with the remote monitoring device 615 through such central controller.

In embodiments, the remote management device 615 evaluates the information received from the wearable unit 691 and provides real time feedback to the user in case any of the body parameters deviate from an expected or reference range. In an embodiment, the system provides real time information to the user through visual/audio/vibration alarms. In another embodiment, the system provides real time information in the form of a dashboard in a hand held device such as the mobile phone or tablet. The user can track such information and in case of any deviation from the standard range of such parameters, the user can take corrective actions. In an embodiment, the corrective actions comprise modifying the movement and or position of a limb to bring the parameters related to such limb in the standard range.

In an embodiment, the information on body parameters captured by various positional sensors is also transmitted to a healthcare professional such as the physician to evaluate the results and recommend corrective action if required. In an embodiment, the system comprises a remote server 620 in data communication with the remote management device 615. The remote server 620 comprises the reference range of parameters against which the body parameters of the user are evaluated.

In various embodiments, the reference ranges of parameters constitute a ‘reference model’ or ‘reference map’ against which the body parameters of the user are evaluated. In some embodiments, the ‘reference model’ or ‘reference map’ is generated by placing one or more wearable units 601 on an unaffected or healthy limb or body portion corresponding to an affected or dysfunctional limb or body portion of a user. For example, consider a case of a patient whose limbs or body portions on one side, say the right side, have been affected due to a stroke or trauma while the corresponding limbs or body portions on the other side, the left side, are unaffected or healthy in terms of body parameters such as posture/movements/gait. In another example, limbs or body portions of a patient, on one side of his body (say, the right side), may be affected by Parkinsonian gait whereas the corresponding limbs or body portion of the patient, on the other side (say, the left side) of the body, may be unaffected and healthy. In such cases, the system 600 is used to generate a ‘reference model’ or ‘reference map’ of the unaffected or healthy limbs or body portions (on the left side, in these examples). The affected or dysfunctional limbs or body portions (on the right side, in these examples) are then monitored, compared and corrected (through real-time feedback and training, for example) with reference to the ‘reference model’ or ‘reference map’. In other words, the ‘reference model’ or ‘reference map’ is generated from the healthy limbs or body portions of the same user who's corresponding other limbs or body portions are affected or dysfunctional.

In some embodiments, the ‘reference model’ or ‘reference map’ is generated by placing one or more wearable units 601 on unaffected or healthy limbs or body portions of a healthy third person (different from the patient whose limbs and/or body portion are affected). For example, consider a case of a patient whose limbs or body portions on both sides of the body have been affected due to stroke or trauma or may be affected by Parkinsonian gait. In such cases, the system 600 is used to generate a ‘reference model’ or ‘reference map’ of the unaffected or healthy limbs or body portions of the healthy third person. The thus generated ‘reference model’ or ‘reference map’ is then used to monitor, compare and correct the posture/movements/gait of the affected or dysfunctional limbs or body portions of the patient.

In various embodiments, the ‘reference model’ or ‘reference map’ is used to provide real-time feedback, training and coaching of healthy individuals such as, but not limited to, dancers, sportspersons, athletes, models, acrobats, gymnasts or other individuals training to acquire specific skills to perform activities that require highly coordinated body movements, posture, position or gait.

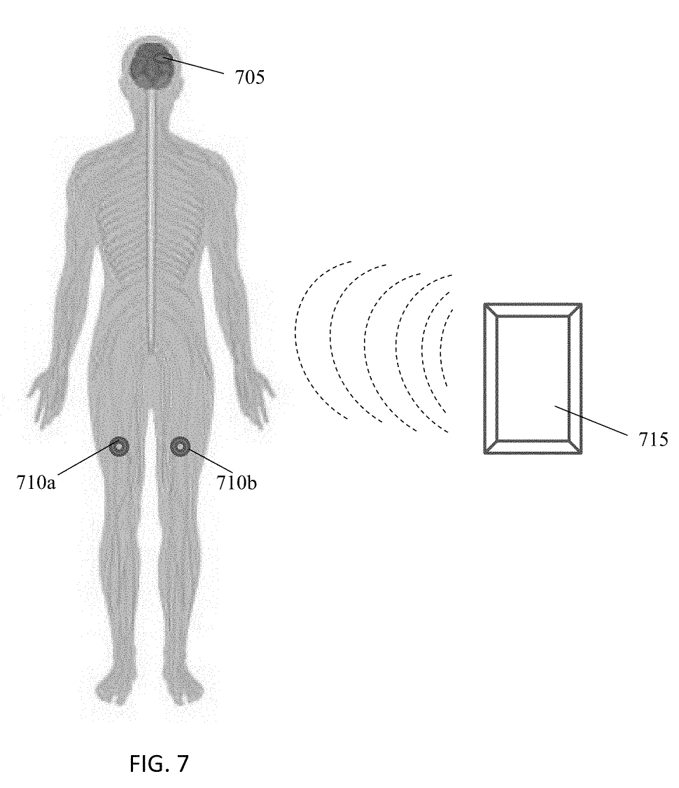

FIG. 7 illustrates bilateral sensors positioned to capture and compare relative movement of the corresponding limbs or body portions in accordance with an embodiment. In an embodiment of the present specification, the user wears sensors 710a and 710b on a first affected or paralytic limb or body portion and a second corresponding unaffected or healthy limb or body portion. Optionally, a head module 705 comprising a sensor to monitor the head posture or movement is also worn by the user. The sensors 705, 710a and 710b capture the body parameters such as posture/movement/gait and transmit this information to the remote management device 715 which evaluates this information and provides real time feedback to the user. In an embodiment, the remote management device 715 uses the body parameters obtained from the second sensor 710b which corresponds to the healthy limb or body portion as reference parameters (reference model′ or ‘reference map’) and compares the body parameters obtained from the sensor 710a which corresponds to the affected limb (impaired model′ or ‘impaired map’) against these reference points. Based on the above comparison, real time feedback is provided to the user.

In another embodiment, the system of the present specification as described in FIG. 7 is used to improve the symmetry between the posture/movement/gait of two corresponding limbs or body portions. In this embodiment, the sensors 710a and 710b are worn or positioned on the two unaffected or healthy limbs or body portions which are required to be symmetrical in terms of posture or movement or gait. People such as fashion models can use this system to improve their walking style by ensuring or enhancing the symmetry between the posture and movement of corresponding body parts such as legs or arms. Further, sportsmen such as swimmers can use this system to ensure that the movement of their arms or legs is symmetrical. In an embodiment, the system compares the information received from the two sensors 710a and 710b to understand the deviation and may recommend changing the posture or movement of both the limbs such that the resultant posture or movement of limbs converges.

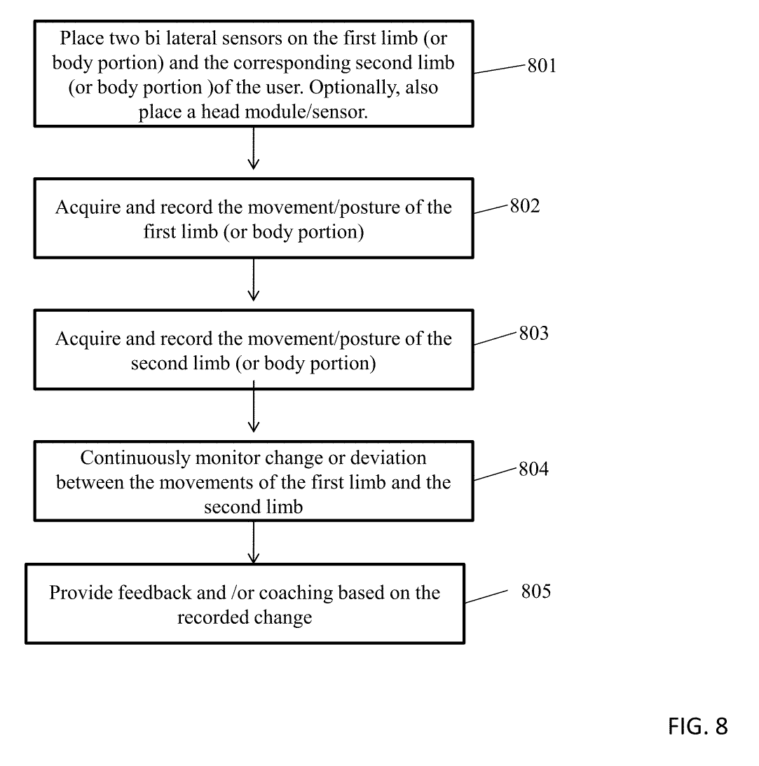

FIG. 8 illustrates a flow chart depicting the steps followed in using the bi lateral sensors positioned on corresponding limbs or body portions in accordance with an embodiment of the present specification. As shown in FIG. 8, at step 801, two bi lateral sensors are positioned on a first limb and a corresponding second limb or body portion of the user. Optionally, a head module comprising a sensor to monitor the movement or posture of head is also positioned on the head of the user. At step 802, the system acquires the body parameters such as the movement/posture/gait corresponding to the first limb from the first sensor. At step 803, the system acquires the similar body parameters corresponding to the second limb from the second sensor. At step 804, the system monitors the deviation in the parameters received for the first limb and the second limb and at step 805 the system provides feedback and/or coaching based on the above comparison.

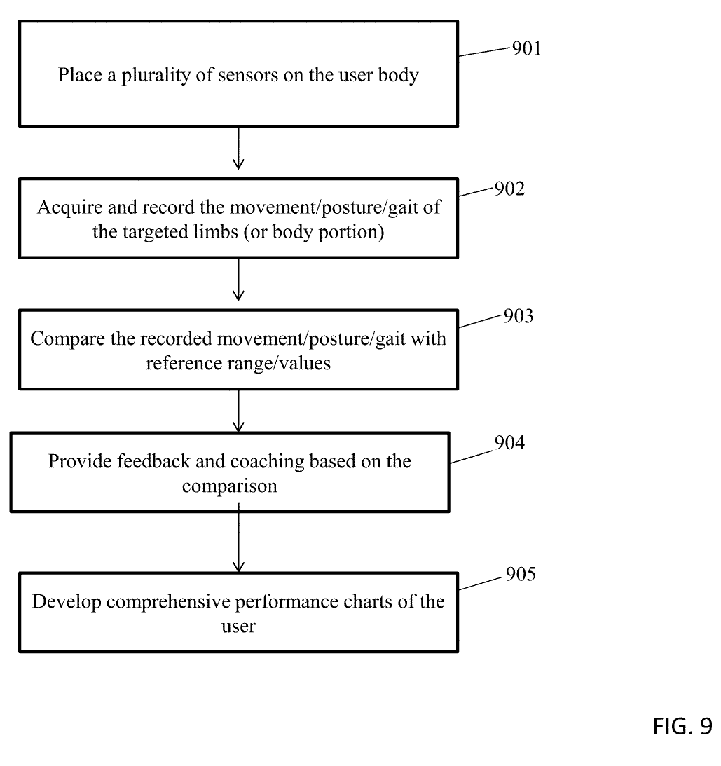

FIG. 9 illustrates a flow chart depicting the steps followed in evaluating the performance of a specific limb or body part against a reference model/map or expected performance level. As shown in FIG. 9, at step 901, a plurality of sensors are placed on the limb or body portion which is required to be monitored. At step 902, the system captures the body parameters such as but not limited to the movement/posture/gait of the target limb or body portion through the sensors placed on such limb or body portion. At step 903, the system compares the captured body parameters with pre-defined and pre-stored reference body parameters (pre-stored ‘reference model or ma’) corresponding to the specific activity which is being monitored. Subsequently, at step 904, the system provides feedback and/or coaching to the user based the above comparison. Optionally, at step 905, the system also develops a performance chart of the user which can be used to evaluate the performance of the user with other users performing a similar activity.

In an exemplary embodiment, the above system can be used by amateur golfers wanting to improve their golf drive or swing. Players spend years in the driving range learning to drive the ball but are usually not able to perfectly master the art. One of the major reasons for this failure is that the posture and the movement of various body parts such as the legs, feet, hips, shoulders, arms plays a critical role in striking a good drive and the players are not able to coordinate all the body parts in the recommended manner. The players never get a proper feedback on which and by how much a specific limb or body part was lagging which led to the underperformance. The system of the present specification can be used wherein positional sensors can be placed on relevant body parts of the player and subsequently the posture/movement/gait of each body part is separately recorded and compared with reference parameters. In an embodiment, the player is provided exact feedback on the performance of each limb or the body part.

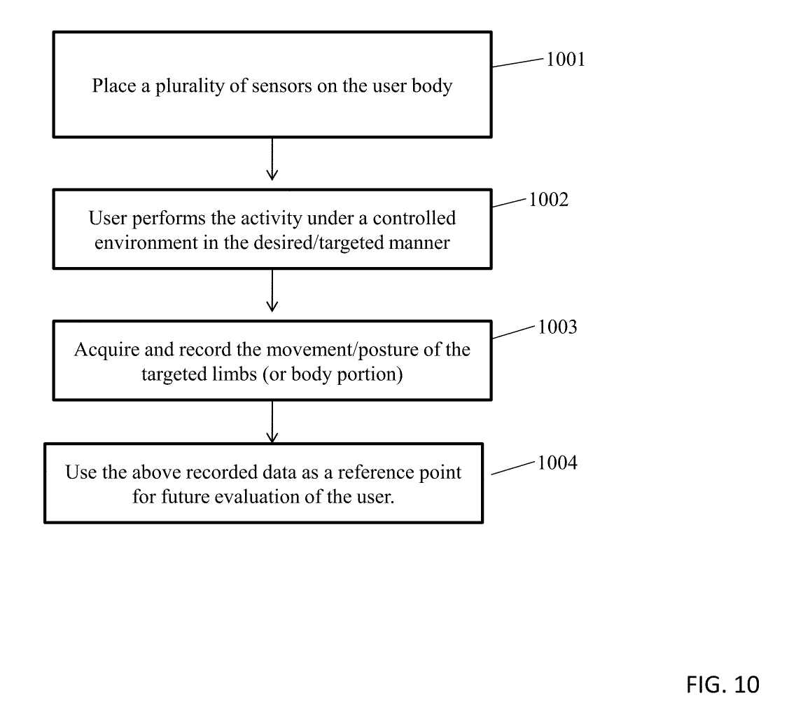

FIG. 10 illustrates a flow chart depicting a plurality of steps followed to use the system, 600 of FIG. 6 in order to estimate a plurality of reference parameters and generate a corresponding ‘reference model’ or ‘reference map’ in a control environment. As shown in FIG. 10, at step 1001, a plurality of sensors are placed on the relevant limbs or body parts of a user. At step 1002, the user performs the required activity under control conditions in a desired/targeted manner. At step 1003, the system 600 captures the various body parameters such as posture/movement/gait and at step 1004, the system 600 stores and uses this information as a reference model, map, level or point for future evaluation. The above method can be used, for example in the game of cricket. A bowler wanting to improve his bowling action may deliver a ball very slowly under the control conditions such that while the speed of delivery might be slow the bowling action is perfect. Subsequently, when the bowler is performing in the actual game, the system 600 can compare his bowling action with the bowling action recorded under the control conditions. The bowler is provided real time information after each ball or a set of balls on his performance with respect to the reference model or map.

Current wearable fitness devices such as Fitbit provide quantitative information such as the number of steps taken to the user. However, these devices do not provide any information on the quality of action such as the body posture and the placement and symmetry between feet while walking. The systems of the present specification provide qualitative information in addition to quantitative information, for example, in embodiments, the number of steps taken with correct posture in addition to the total number of steps taken. In an embodiment, the system of the present specification is configured such that the information received from various positional sensors is stored on a remote server from which it can be accessed through other wearable fitness devices such as Fitbit or Jawbone to perform a complete analysis and provide a holistic feedback to the user.

The above examples are merely illustrative of the many applications of the methods and systems of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for improving a motor function of a patient after said patient has a stroke, comprising:
positioning at least one first stimulation unit on the patient, wherein said at least one first stimulation unit is placed in electrical communication with a first region of the patient, said first region comprising a plurality of muscles requiring improvement of said motor function;
acquiring, via a controller located in a mobile device external to said at least one first stimulation unit, first signals generated by said at least one first stimulation unit, said first signals representative of a plurality of body parameters associated with said first region of the patient;
comparing said first signals with reference signals; and,
performing at least one of the following actions if said first signals deviate from said reference signals beyond a threshold:
activating said at least one first stimulation unit to apply electrical stimulation to said first region and,
triggering feedback for the patient.

2. The method of claim 1, wherein said plurality of body parameters comprise data representative of at least one of position, posture, gait and movement of said first region of the patient.

3. The method of claim 1, wherein said reference signals are acquired by said controller from at least one second stimulation unit placed in electrical communication with a plurality of muscles not requiring improvement of said motor function in a second region of the patient.

4. The method of claim 3, wherein said first region is a limb on a right side of the patient's body, and wherein said second region is a corresponding limb on a left side of the patient's body.

5. The method of claim 3, wherein said first region is a limb on a left side of the patient's body, and wherein said second region is a corresponding limb on a right side of the patient's body.

6. The method of claim 1, wherein said reference signals are generated from at least one second stimulation unit placed in electrical communication with a plurality of muscles in a second region of a healthy person, wherein said healthy person is an individual with muscles that do not have an impaired motor function.

7. The method of claim 6, wherein said first region is a limb on a right side of the patient's body, and wherein said second region is a corresponding limb on a right side of the healthy person's body.

8. The method of claim 6, wherein said first region is a limb on a left side of the patient's body, and wherein said second region is a corresponding limb on a left side of the healthy person's body.

9. The method of claim 1 further comprising: positioning a head stimulation unit on a head of the patient, wherein the head stimulation unit is placed in electrical communication with a first portion of a brain of the patient.

10. The method of claim 9 wherein the at least one first stimulation unit and the head stimulation unit are activated synchronously.

11. The method of claim 9 wherein the at least one first stimulation unit and the head stimulation unit are activated asynchronously.

12. The method of claim 9 wherein said plurality of muscles are in a limb in a right side of the patient's body.

13. The method of claim 12 wherein said first portion is in a left side of the brain.

14. The method of claim 12 wherein said first portion is in a right side of the brain.

15. The method of claim 9 wherein said plurality of muscles are in a limb in a left side of the patient's body.

16. The method of claim 15 wherein said first portion is in a right side of the brain.

17. The method of claim 15 wherein said first portion is in a left side of the brain.

18. A method for improving a function of a patient, comprising:
positioning at least one first stimulation unit on the patient, wherein said at least one first stimulation unit is placed in electrical communication with a first region of the patient, said first region comprising at least one limb of said patient;
acquiring, via a controller located in a mobile device external to said at least one first stimulation unit, first signals generated by said at least one first stimulation unit, said first signals representative of a reference model or map of said first region of the patient, wherein said reference model or map defines a baseline position or movement of said first region and said first signals are acquired while said first region is in a first state;
acquiring, via the controller, second signals generated by said at least one first stimulation unit, wherein said second signals are acquired while said first region performs said function;
comparing said first signals with second signals to determine a deviation of position or movement of said first region from said baseline position or movement; and
performing at least one of the following actions if said first signals deviate from said second signals beyond a threshold:
activating said at least one first stimulation unit to apply electrical stimulation to said first region and
triggering a feedback for the patient.

19. The method of claim 18, wherein said first state is representative of said first region performing said function, and wherein said function is at least one of a reference position, posture, gait and movement of said first region.

20. The method of claim 18, further comprising: positioning a second stimulation unit on a head of the patient, wherein the second stimulation unit is placed in electrical communication with a first portion of a brain of the patient.

21. The method of claim 20 wherein the first stimulation unit and second stimulation unit are activated synchronously.

22. The method of claim 20 wherein the first stimulation unit and second stimulation unit are activated asynchronously.

23. The method of claim 18 wherein the at least one limb comprises a plurality of muscles requiring a restoration of function after said patient has one of a stroke and a Parkinsonian gait.

24. The method of claim 18 wherein the at least one limb comprises a healthy limb.

25. The method of claim 20 wherein said first region is in a right side or a left side of the patient's body and said first portion is in a right side or a left side of the brain.
